(19) United States
(12) Patent Application Publication
(10) Pub. No.: US 2016/0060708 A1
(43) Pub. Date: Mar. 3, 2016

(54) Method for detecting the risk of early gastric cancer

(71) Applicant: Taichung Veterans General Hospital, Taichung (TW)

(72) Inventor: Chun-Ying Wu, Taichung (TW)

(21) Appl. No.: 14/871,626

(22) Filed: Sep. 30, 2015

Related U.S. Application Data
(63) Continuation-in-part of application No. 14/034,750, filed on Sep. 24, 2013.

(30) Foreign Application Priority Data
Mar. 19, 2013 (TW) 102109695

(51) Int. Cl. C12Q 1/68

(52) U.S. Cl. CPC C12Q 1/6886; C12Q 2600/158

(57) ABSTRACT

The present invention discloses the nine biomarkers including HIF1A, FAM84B, CRIP2, GSN, RPL15, DLG1, MAT2A, PGBD2 and ID3 are respectively selected according to their specific and unique expression profile in the gastric cancer cells or gastric cancer tissue. Therefore, the nine biomarkers are related to diagnose gastric cancer, such as detecting early gastric cancer, staging gastric cancer, predicting prognosis of gastric cancer and diagnosing lymph node metastasis. By analyzing the expression value of at least one biomarker of a sample from a subject, the subject can be precisely diagnose the risk about gastric cancer.

The current application is a continuation-in-part of 14/034,750 filed on Sep. 24, 2013. The current application claims a foreign priority to application No. 102109695 filed on Mar. 19, 2013 in Taiwan.

### FIELD OF INVENTION

The present invention relates generally to detection of cancer, and more specifically the invention is related to a method for detecting the risk of early gastric cancer.

### BACKGROUND OF INVENTION

Gastric cancer is the forth most common malignancy in the worldwide according to the statistic by the statistics in WHO. Especially, gastric cancer is also considered as the severe neoplasm due to its role in the second most common leading cause of cancer deaths. Herein, the mortality of gastric cancer in Asia is higher than that in Europe and USA. In addition, Japan is the country has the highest incidence of gastric cancer in the worldwide. Recently, the incidence and mortality of gastric cancer is lowing by the newly proposed guidances in healthy concepts and the improved dietary habits. However, the reductions in incidence and mortality of gastric cancer in the Asian countries such as Taiwan and Japan are not obvious. In Taiwan, gastric cancer is the fifth leading cause of cancer deaths that includes 1482 cases in male and 806 in female according to the statistics by Department of Health in 2011.

Gastric cancer is a multivariate malignancy that can be classified into early gastric cancer and late gastric cancer (or advanced gastric cancer) by the invasion degree of cancer cells. Herein, the cancer cells of the early gastric cancer exhibit the invasion into gastric mucosa and submucosa layers. The patients bearing early gastric cancer reveal 5-years survival up to 95% after surgery. Conversely, the cancer cells of late gastric cancer usually invade into the muscle layer and serosa layer, and result in the drastic reduction of the 5-years survival rate after surgery. However, the disfunction of the regional mucosa layer resulted from the thicken stomach wall, which is the symptom in the early gastric cancer, is too mild to be found. In other words, there is no specific symptom for early gastric cancer progression to warn the patient for adopting the further physical examination. The associated symptoms in gastric cancer patients such as vomiting, poor appetite, dyspepsia, and diarrhea are difficult to be distinguished from the other disorders occurred in the digestion system, which cause late detection of gastric cancer in clinical, so that the 5-years survival rate of the patient is less than 50%. Therefore, the efficient method for detecting gastric cancer and correct staging gastric cancer before surgery is the critical issues for improving the survival of patients.

Further, the current diagnosing approach for gastric cancer is gastroscopy that contains several disadvantages such as the poor acceptability for patients. In addition, the gastroscopy diagnosis requires large attention, long time spent and expensive cost. These disadvantages of gastroscopy suggest the requirement of the newly developed diagnosis method with greater acceptability and better benefit. The lacking of the appropriated diagnosis in clinical leads to that the gastric cancer in more than 80% of patients are found at advanced stage and causes the poor survival rate. Moreover, the complete resection of the tumor and the metastasized lymph nodes is the most efficient treatment in clinical. Therefore, staging of the gastric cancer upon the non-invasive image detection systems such as CT and MRI are required for improving the patient survival rate before surgery. But it is difficult to identify that the lymph node metastasis or the metastasized organ is smaller than 5 mm, which makes more than 50% gastric cancer patients can't be correctly preoperative staging and limits the improvement of cure rate.

The resent studies suggest that the whole genome sequencing is approached for investigating the genome, transcriptomes, and epigenome of the cancer cells to examine the phenotypes in patients. In addition, the correlation of the genetic analysis and clinical investigations provides the useful informations for the clinical researches and management. However, the recent cost for whole genome sequencing is too expansive to be applied in clinical.

Otherwise, some studies identify the new biomarkers for gastric cancer diagnosis by the RNA-based global gene expression strategy analysis. For example, the expression of osteopontin (OPN) is applied as the potential biomarker for predicting the invasion of gastric cancer. The compared expression profile of the gastric cancer cells and the adjacent normal region assessed by microarray analysis also shows the over-expression of OPN in the gastric cancer cells. In the gastric cancer cell line with highly metastatic potency, the transforming liver-metastasis gastric cancer cells exhibit 2.7˜10.2 folds of OPN expression greater than that in parental cells. Collectively, the increased OPN expression in the plasma is positively correlated with occurrence and invasiveness of gastric cancer, and the survival rate of patient according to the studies using RNA-based global gene expression strategy analysis.

Moreover, the previous studies using microarray analysis also suggest that some members in matrix metalloproteinases (MMPs) family involve in the molecular regulation of the gastric cancer progression. Herein, the result of cDNA microarray shows that the MMP-9 expression detected from the plasma is more reliable for precisely predicting the occurrence and progression of gastric cancer than the MMP-9 expression in serum. The previous studies also reveal that the expressions of MMP-2 and tissue inhibitor of metalloproteinase-2 (TIMP-2) are related with the invasiveness of gastric cancer but dispensable for gastric cancer progression.

Taken together, some biomarkers for gastric cancer diagnosis and staging have been currently developed. However, these biomarkers still lack the high specificity and high sensitivity for gastric cancer diagnosis and staging. For example, the prediction of the gastric cancer occurrence upon OPN expression reveals the accuracy up to 63.6%. Furthermore, the diagnosis accuracy of serosa layer invasion and liver metastasis according to the OPN expression in gastric cancer patients are 62.9% and 83%, respectively. In addition, diagnosis sensitivity and specificity of gastric cancer by the MMP-9 expression in the plasma are 82% and 65.5%, respectively.

The expressions of these indicated biomarkers for gastric cancer diagnosis are interfered by many physiological conditions in the patients. For example, the expression value of OPN is affected by age, hyperlipidemia, cardiovascular diseases, renal disease, diabetes and pyemia. The elevated OPN expression value in the plasma caused by these indicated physiological conditions will lead to misjudgment in clinical. In addition, applied biomarkers without exclusion of the exogenous factors such as drug or helicobacter pylori infection cannot provide the great diagnosis accuracy of gastric cancer progression.

Currently, the lacking of the method for precisely detecting early gastric cancer or correctly preoperative staging gastric cancer limits the improvements of the curing rate and identification of gastric cancer. Therefore, the development of the method for detecting early gastric cancer and correct staging gastric cancer is quite important to improve the public health.

### SUMMARY OF INVENTION

The present invention provides a method for detecting the risk of early gastric cancer, comprising the following steps:

(a) providing at least one biological sample from a subject with gastric cancer and at least one biological sample from a subject without gastric cancer;

(b) measuring the expression of at least one biomarker in the biological samples, wherein the biomarker is CRIP2;

(c) analyzing the expressions of the biomarker obtained in step b by regression analysis and drawing a receiver operating characteristic (ROC) curve to obtain a cut-off value;

(d) measuring the expression of the biomarker in a sample from a test subject; and

(e) comparing the expression of the biomarker in the sample from the test subject with the cut-off value of step c to predict the risk of the test subject having early gastric cancer, wherein when the expression value of the biomarker of the test subject below the cut-off value is indicative of a higher risk of the presence of early gastric cancer in the test subject.

In the embodiments, the sample can be a blood specimen, a cell of stomach wall or a tissue of stomach wall. Preferably, the sample is the blood specimen.

In one embodiment, the biomarker of step b further includes a gene selected from the group consisting of FAM84B, RPL15, DLG1, MAT2A, PGBD2 and ID3.

In another embodiment, the biomarker is further spotted on a matrix. Preferably, the matrix is a microarray.

In the other embodiment, in the method, the step b is further to measure the expression of another biomarker: HIF1A; the step c is to analyze the expressions of the another biomarker obtained in step b by regression analysis and drawing another ROC curve to obtain another cut-off value; the step d is to measure the expression of the another biomarker in the sample from the test subject; and the step e is to compare the expression of the another biomarker in the sample from the test subject with the another cut-off value of step c to predict the risk of the test subject having early gastric cancer, wherein when the expression value of the another biomarker of the test subject above the another cut-off value is indicative of a higher risk of the presence of early gastric cancer in the subject.

According the above method, it can precisely predict the risk of the present of early gastric cancer regardless of the different combinations and number of the biomarkers. Furthermore, Comparing with the gastroscopy of the prior art, it can be more convenient and acceptable to a subject or a patient by using the blood tissue as the sample.

### BRIEF DESCRIPTION OF THE DRAWINGS

FIG. 1 shows the ROC curve for the biomarkers of HIF1A and PGBD2.

FIG. 2 shows the ROC curve of the biomarkers of HIF1A, PGBD2 and FAM84B.

FIG. 3 shows the ROC curve of the biomarkers of HIF1A, PGBD2 and CRIP2.

FIG. 4 shows the ROC curve of the biomarkers of HIF1A, PGBD2 and RPL15.

FIG. 5 shows the ROC curve of the biomarkers of HIF1A, PGBD2 and DLG1.

FIG. 6 shows the ROC curve of the biomarkers of HIF1A, PGBD2 and MAT2A.

FIG. 7 shows the ROC curve of the biomarkers of HIF1A, PGBD2 and ID3.

FIG. 8 shows the ROC curve of the biomarkers of HIF1A, FAM84B and ID3.

FIG. 9 shows the ROC curve of the biomarkers of HIF1A, PGBD2, CRIP2 and DLG1.

FIG. 10 shows the ROC curve of the biomarkers of CRIP2, DLG1 and MAT2A.

FIG. 11 shows the ROC curve of the biomarkers of FAM84B, CRIP2, DLG1 and MAT2A.

FIG. 12 shows the ROC curve of the risk score calculated assessed by the formula

FIG. 13 shows the Kaplan-Meier survival curve for the patients with different risk ranking.

FIG. 14 shows the Kaplan-Meier survival curve for the patients with different expression value of the biomarker of FAM84B.

### DETAILED DESCRIPTION OF THE INVENTION

The details of one or more embodiments of the invention are set forth in the accompanying description below.

The invention is based on a discovery of a novel gastric cancer biomarker such as HIF1A, FAM84B, CRIP2, GSN, RPL15, DLG1, MAT2A, PGBD2 or ID3 identified according to its specific and unique expression profile in gastric cancer cells or gastric cancer specimens. Hence, in one aspect, the invention provides each one of the biomarkers or a combination of the biomarkers to diagnose gastric cancer.

Furthermore, the inventor established the isogenic invasion subclones from Human gastric cancer cell line (AGS cells) in the Matrigel invasion chambers for investigations including migration assay and colony-forming assay. Following, nine candidate genes revealing positive expressing difference are acquired by the transcriptomes comparison between the AGS cells and the passaged subclone cells. The identified nine candidates are the present biomarkers for gastric cancer diagnosis in the invention.

The invention also provides a method for diagnosis gastric cancer, comprising the following steps: (a) providing a biological sample from a subject; (b) measuring the expression value of at least one biomarker in the biological sample; and (c) analyzing the expression value of the biomarker measured in the step b to determine the process of gastric cancer or the occurrence of early gastric cancer in the subject. Herein, the biological sample can be a blood tissue specimen, cells and histological sections of stomach wall, or any suitable tissue. In the following description, using the blood specimen is only exemplary and illustrative, not limiting in scope.

Furthermore, in the following detailed description of the invention, using different statistic models and tests in the SAS software to analyze data is merely exemplary and is not to limit the invention to the forms disclosed.

In the other aspect, the invention provides a diagnosis kit or a biochip for diagnosing gastric cancer by spotted at least one biomarker on a microarray. Herein, the manufactured chip processed by micro-electro technology or other processing technologies that spot at least one kind of bio-probe on a platform made of glass, silicon or high molecular weight material for the diagnosis and medical detection kit. According to the hybridization of DNA probe with oligonucleotides or the specific binding of protein probe with detecting proteins in the specimens, the microarray is used for detecting the specific candidates in the specimens. For example, DNA microarray is spotted with oligonucleotides as the detecting probe and the protein chip is spotted with protein as the detecting probe. Therefore, in the invention, the gastric cancer biomarkers and their transcripts can be spotted on the microarray as the probes to detect the biomarkers expression in the specimens.

Unless defined otherwise, all technical and scientific terms used herein have the same meanings commonly understood by one of ordinary skill in the art. As used in this application, including claims, the following words or phases have the meanings specified.

The term of “diagnose gastric cancer” or “diagnosis gastric cancer” refers to detection early gastric cancer, staging gastric cancer, diagnosis lymph node metastasis, prognosis or post-surgery survival rate.

The term “Student-t test” is used to determine whether two sets of data in normal distribution are significantly different to each other.

The term “Chi-square test” is used to determine the significant correlation between two parameters. The term “Mann-Whitney U test” is used for analyzing the difference between the medians of two groups.

The term “Wilcoxon rank sum test” is used for comparing the distributions of two sets of data.

The term “Chi-square test with Yate's continuity correction” refers to when the expected frequency value is between 5 to 10, the result of Chi-square shall be with Yate's continuity correction.

The term “ANOVA test” is used to examine whether the average of three or more than three groups are equal or not.

The term “Kruskal-Wallis Test” is used to examine whether the medians of three or more than three groups are equal or not. The term “Fisher's exact test” is for categorical data resulted from classifying objects in two different ways. It is usually used for analyzing the objects with small sample size.

The term “Kaplan-Meier estimator” refers to an estimator to calculate the survival curve.

The term “Log-Rank test” refers to a test to examine whether the difference between two survival curves is statistically significant.

The term “mean±SD” refers to average ±standard deviation.

The term “Median” refers to a numerical value that separates the higher and lower half of a set of numbers.

The term “AUC” refers to the aberration of area under curve that means the area under ROC curve.

There are 129 samples used in the following examples for characterizing the expression of the biomarkers by the biomedicine examination technologies. Each sample is Buffy coats from the blood specimen randomly collected from the patients with surgery resection for gastric cancer or health examination in Taichung Veterans General Hospital from December 2007 to December 2010, wherein the patients had not received adjuvant chemotherapy. Totally, 44 samples were collected from the patients with gastric cancer and 85 samples were collected from the patients without gastric cancer. The collected samples and the following examples are in conformity to bioethical constraints, including obtaining the approval of the institutional review board of Taichung Veterans General Hospital and the informed consent from the patients.

The age, sex, occurrence and stage of gastric cancer, recurrence, recurrent interval and lymph node metastasis are shown in table 1, wherein P value with the marker § was determined using Student T-test and P value with the marker # was determined using chi-square test.

TABLE 1
the data of the subjects.

| | | Normal | Gastric cancer | | |
|---|---|---|---|---|---|
| | | (n = 85) | (n = 44) | | p value |
| Age | mean ± SD | 57.79 ± 5.15 | 59.16 ± 5.88 | | 0.1716^{§} |
| Sex | Female | 42 (48.28%) | 19 | (43.18%) | 0.5809^{#} |
| | Male | 45 (51.72%) | 25 | (56.82%) | |
| Stage | Stage 1 | | 8 | (18.18%) | |
| | Stage 2 | | 6 | (13.64%) | |
| | Stage 3 | | 11 | (25.00%) | |
| | Stage 4 | | 19 | (43.18%) | |
| Lethal | No | | 36 | (81.82%) | |
| | Yes | | 8 | (18.18%) | |

| | | |
|---|---|---|
| Observing | mean ± SD | 541.64 ± 325.83 |
| period | | |
| (Unit: day) | | |

| | | | |
|---|---|---|---|
| Cancer | No | 37 | (84.09%) |
| recurrence | Yes | 7 | (15.91%) |

| | | |
|---|---|---|
| Latency of | mean ± SD | 224.14 ± 123.70 |
| recurrence | | |

| | | | |
|---|---|---|---|
| Lymph node | No | 13 | (29.55%) |
| metastasis | Yes | 31 | (70.45%) |

In table 1, the statistic P values higher than 0.05 suggest the nonsignificant association between the occurrence of gastric cancer with the age and sex in the subjects.

### EXAMPLE 1

### Examination of the Expression Values of the Nine Biomarkers in Each Sample

The total RNA was extracted from the sample collection treated with TRIzol® reagent (Invitrogen, Carlsbad, Calif., USA) for the quantitative RT-PCR (qRT-PCR) to characterize the expression values of the nine biomarkers for gastric cancer. In the reverse transcription, the extracted total RNA reverse transcribed to cDNA with Advantage RT-for-PCR kit (Clontech, USA).

After synthesizing first strand of cDNA, the expression values of the biomarkers were determined by quantitative real time PCR with FastStart Universal Probe Master Rox reagent (Roche). The reverse primers and Universal ProbeLibrary probes were chosen as suggested by Roche Universal Probe library. Finally, the expression values of the biomarkers were measured using the ABI StepOnePlus Real-Time PCR System (Applied Biosystem). The sets of primers used for the quantified real time PCR are listed in table 2.

TABLE 2
The primer sets of the biomarkers.

| Biomarker | Primer |
|---|---|
| CRIP2 | SEQ ID No. 1 [symbol] BSEQ ID No. 2 |
| DLG1 | SEQ ID No. 3 [symbol] BSEQ ID No. 4 |
| FAM84B | SEQ ID No. 5 [symbol] BSEQ ID No. 6 |
| GSN | SEQ ID No. 7 [symbol] BSEQ ID No. 8 |
| HIF1A | SEQ ID No. 9 [symbol] BSEQ ID No. 10 |
| ID3 | SEQ ID No. 11 [symbol] BSEQ ID No. 12 |
| MAT2A | SEQ ID No. 13 [symbol] BSEQ ID No. 14 |
| PGBD2 | SEQ ID No. 15 [symbol] BSEQ ID No. 16 |
| RPL15 | SEQ ID No. 17 [symbol] BSEQ ID No. 18 |

For each reaction, the total reaction volume was 20 μl containing 10 μl of FastStart Universal Probe master Rox reagent (Roche), 0.4 μl of each primer with concentration 10 μM, 0.2 μl hydrolysis probe and with 50, 25, 6.25, 3.125, 0.7813, 0.3906 ng of cDNA. Cycling condition were 50° C. for 2 minutes, 95° C. for 10 minutes, 40 cycles of 95° C. for 15 seconds and 60° C. for 1 minute.

### EXAMPLE 2

### Each Biomarker for Detection Gastric Cancer

The expression values of the nine biomarkers from the samples characterized by the quantitative real time PCR were further statistically analyzed by Mann-Whitney U test. The P value of the statistic data shown in table 3 was determined using Wilcoxon rank sum test.

TABLE 3
Mann-Whitney U test results for each biomarker

| | | | | Wilcoxon | |
|---|---|---|---|---|---|
| | | Normal | Gastric cancer | rank sum | |
| | | (n = 87) | (n = 44) | test | P value |
| HIF1A | mean ± | 0.80 ± 0.44 | 1.93 ± 1.29 | | |
| | SD | | | | |
| | median | 0.77 | 1.66 | 4107.0 | <0.0001 |
| FAM84B | mean ± | 0.17 ± 0.14 | 0.05 ± 0.06 | | |
| | SD | | | | |
| | median | 0.13 | 0.03 | 1508.5 | <0.0001 |
| CRIP2 | mean ± | 6.18 ± 3.96 | 2.10 ± 2.22 | | |
| | SD | | | | |
| | median | 5.83 | 1.47 | 1549.0 | <0.0001 |
| GSN | mean ± | 1.83 ± 1.31 | 3.55 ± 2.29 | | |
| | SD | | | | |
| | median | 1.57 | 3.11 | 3950.5 | <0.0001 |
| RPL15 | mean ± | 3.21 ± 1.32 | 1.39 ± 0.84 | | |
| | SD | | | | |
| | median | 3.19 | 1.18 | 1394.0 | <0.0001 |
| DLG1 | mean ± | 1.33 ± 0.59 | 0.61 ± 0.39 | | |
| | SD | | | | |
| | median | 1.26 | 0.56 | 1580.5 | <0.0001 |
| MAT2A | mean ± | 1.44 ± 0.60 | 0.67 ± 0.41 | | |
| | SD | | | | |
| | median | 1.41 | 0.56 | 1530.0 | <0.0001 |
| PGBD2 | mean ± | 3.76 ± 1.92 | 1.32 ± 0.77 | | |
| | SD | | | | |
| | median | 3.56 | 1.17 | 1378.5 | <0.0001 |
| ID3 | mean ± | 1.00 ± 0.59 | 0.28 ± 0.24 | | |
| | SD | | | | |
| | median | 0.88 | 0.21 | 1253.0 | <0.0001 |

In table 3, the p value of each biomarker less than 0.005 suggests that the expression value of each biomarker in the gastric cancer patients is significantly different to that in the normal sample providers. Therefore, each biomarker is capable of application in gastric cancer prediction in clinical. Moreover, the expression value of each biomarker was further analyzed by logistic regression analysis from SAS software. The analyzed results were shown in table 4 as below.

TABLE 4
The univariate logistic regression results of each biomarker

| | | | 95% confidence | | | | |
|---|---|---|---|---|---|---|---|
| | | | interval | | | | |
| | | Odds | Lower | Upper | | | Cut off |
| | β-estimate | ratio | bound | bound | P value | AUC | value |
| HIF1A | 2.0880 | 8.069 | 3.503 | 18.587 | <0.0001 | 81.4% | 0.93 |
| FAM84B | −22.3014 | <0.001 | <0.001 | <0.001 | <0.0001 | 86.4% | 0.05 |
| CRIP2 | −0.5091 | 0.601 | 0.487 | 0.741 | <0.0001 | 85.4% | 2.73 |
| GSN | 0.6045 | 1.830 | 1.393 | 2.405 | <0.0001 | 77.3% | 2.64 |
| RPL15 | −1.7733 | 0.170 | 0.090 | 0.321 | <0.0001 | 89.5% | 2.14 |
| DLG1 | −3.0644 | 0.047 | 0.015 | 0.147 | <0.0001 | 84.6% | 0.96 |
| MAT2A | −2.9079 | 0.055 | 0.019 | 0.158 | <0.0001 | 85.9% | 1.08 |
| PGBD2 | −1.6796 | 0.186 | 0.097 | 0.357 | <0.0001 | 89.9% | 2.34 |
| ID3 | −6.8415 | 0.001 | <0.001 | 0.012 | <0.0001 | 93.1% | 0.37 |

The table 4 shows that the accuracy of each biomarker for detecting the occurrence of gastric cancer is more than 70%. Herein, the accuracy of detecting gastric cancer by HIF1A is 81.4%; the accuracy of detecting gastric cancer by FAM84B is 86.4%; the accuracy of detecting gastric cancer by CRIP2 is 85.4%; the accuracy of detecting gastric cancer by GSN is 77.3%; the accuracy of detecting gastric cancer by RPL15 is 89.5%; the accuracy of detecting gastric cancer by DLG1 expression is 84.6%; the accuracy of detecting gastric cancer by MAT2A is 85.9%; the accuracy of detecting gastric cancer by PGBD2 expression is 89.9%; and the accuracy of detecting gastric cancer by ID3 expression is 93.1%.

Furthermore, each biomarker was divided into two groups by its cut-off value for assess by logistic regression in SAS software. The assessed results were shown in table 5, wherein the p value was determined using Chi-square test with Yate's continuity correction.

TABLE 5
The univariate logistic regression result for the two
groups of each biomarker divided by its cut off value

| | | Normal | Gastric cancer | | Odd ratio | | |
|---|---|---|---|---|---|---|---|
| | Cut off | (n = 87) | (n = 44) | | (95% confidence | | P |
| | value | Number (%) | Number (%) | | interval) | AUC | value |
| HIF1A | ≦0.93 | 59 (67.82) | 7 | (15.91) | 1 | | |
| | >0.93 | 28 (32.18) | 37 | (84.09) | 11.1378 (4.4182, | 76.0% | <0.0001 |
| | | | | | 28.0771) | | |
| FAM84B | ≦0.05 | 11 (12.64) | 32 | (72.73) | 1 | | |
| | >0.05 | 76 (87.36) | 12 | (27.27) | 0.0543 (0.0217, | 80.0% | <0.0001 |
| | | | | | 0.1357) | | |
| CRIP2 | ≦2.73 | 15 (17.24) | 34 | (77.27) | 1 | | |
| | >2.73 | 72 (82.76) | 10 | (22.73) | 0.0613 (0.0250, | 80.0% | <0.0001 |
| | | | | | 0.1504) | | |
| GSN | ≦2.61 | 74 (85.06) | 16 | (36.36) | 1 | | |
| | >2.61 | 13 (14.94) | 28 | (63.64) | 9.9615 (4.2522, | 74.3% | <0.0001 |
| | | | | | 23.3366) | | |
| RPL15 | ≦2.14 | 22 (25.29) | 38 | (86.36) | 1 | | |
| | >2.14 | 65 (74.71) | 6 | (13.64) | 0.0534 (0.0199, | 80.5% | <0.0001 |
| | | | | | 0.1435) | | |
| DLG1 | ≦0.96 | 24 (27.59) | 38 | (86.36) | 1 | | |
| | >0.96 | 63 (72.41) | 6 | (13.64) | 0.0602 (0.0226, | 79.4% | <0.0001 |
| | | | | | 0.1604) | | |
| MAT2A | ≦1.08 | 27 (31.03) | 35 | (79.55) | 1 | | |
| | >1.08 | 60 (68.97) | 9 | (20.45) | 0.1157 (0.0489, | 74.3% | <0.0001 |
| | | | | | 0.2740) | | |
| PGBD2 | ≦2.34 | 24 (27.59) | 41 | (93.18) | 1 | | |
| | >2.34 | 63 (72.41) | 3 | (6.82) | 0.0279 (0.0079, | 82.8% | <0.0001 |
| | | | | | 0.0986) | | |
| ID3 | ≦0.37 | 5 (5.75) | 35 | (79.55) | 1 | | |
| | >0.37 | 82 (94.25) | 9 | (20.45) | 0.0157 (0.0049, | 86.9% | <0.0001 |
| | | | | | 0.0501) | | |

The statistic results shown in table 5 suggest that the accuracy of detecting early gastric cancer by the expression value of each biomarker is more than 70%, wherein the accuracy of the prediction for gastric cancer by HIF1A expression is 76.0%; the accuracy of the prediction for gastric cancer by FAM84B is 80.0%; the accuracy of the prediction for gastric cancer by CRIP2 is 80.0%; the accuracy of the prediction for gastric cancer by GSN is 74.3%; the accuracy of the prediction for gastric cancer by RPL15 is 80.5%; the accuracy of the prediction for gastric cancer by DLG1 is 79.4%; the accuracy of the prediction for gastric cancer by MAT2A is 74.3%; the accuracy of the prediction for gastric cancer by PGBD2 is 82.8%; and the accuracy of the prediction for gastric cancer by ID3 is 86.9%.

In detail, a sample provider with HIF1A expression value higher than 0.93 shows 11.1378 folds of risk of acquiring gastric cancer with comparison of a sample provider with HIF1A expression value equal or less than 0.93; A sample provider with FAM84B expression value higher than 0.05 shows 0.0543 fold of risk of acquiring gastric cancer with comparison of a sample provider with FAM84B expression value equal or less than 0.05; a sample provider with CRIP2 expression value higher than 2.73 shows 0.0613 fold of risk of acquiring gastric cancer with comparison of a sample provider with CRIP2 expression value equal or less than 2.73; a sample provider with GSN expression value higher than 2.61 shows 9.9615 folds of risk of acquiring gastric cancer with comparison of a sample provider with GSN expression value equal or less than 2.61; a sample provider with RPL15 expression value higher than 2.14 shows 0.0534 fold of risk of acquiring gastric cancer with comparison of a sample provider with RPL15 expression value equal or less than 2.14; a sample provider with DLG1 expression value higher than 0.96 shows 0.0602 fold of risk of acquiring gastric cancer with comparison of a sample provider with DLG1 expression value equal or less than 0.96; a sample provider with MAT2A expression value higher than 1.08 shows 0.1157 fold of risk of acquiring gastric cancer with comparison of a sample provider with MAT2A expression value equal or less than 1.08; a sample provider with PGBD2 expression value higher than 2.34 shows 0.0279 fold of risk of acquiring gastric cancer with comparison of a sample provider with PGBD2 expression value equal or less than 2.34; and a sample provider with ID3 expression value higher than 0.37 shows 0.0157 fold of risk of acquiring gastric cancer with comparison of a sample provider with ID3 expression value equal or less than 0.37.

According to table 5, it suggests that the sample provided from the patient bearing gastric cancer reveals increased expression value of HIF1A or GSN. In contrast, the expression value of FAM84B, CRIP2, RPL15, DLG1, MAT2A, PGBD2 or ID3 is decreased in the sample collected from the gastric cancer patient.

### EXAMPLE 3

### HIF1A and PGBD2 for Diagnosis Gastric Cancer

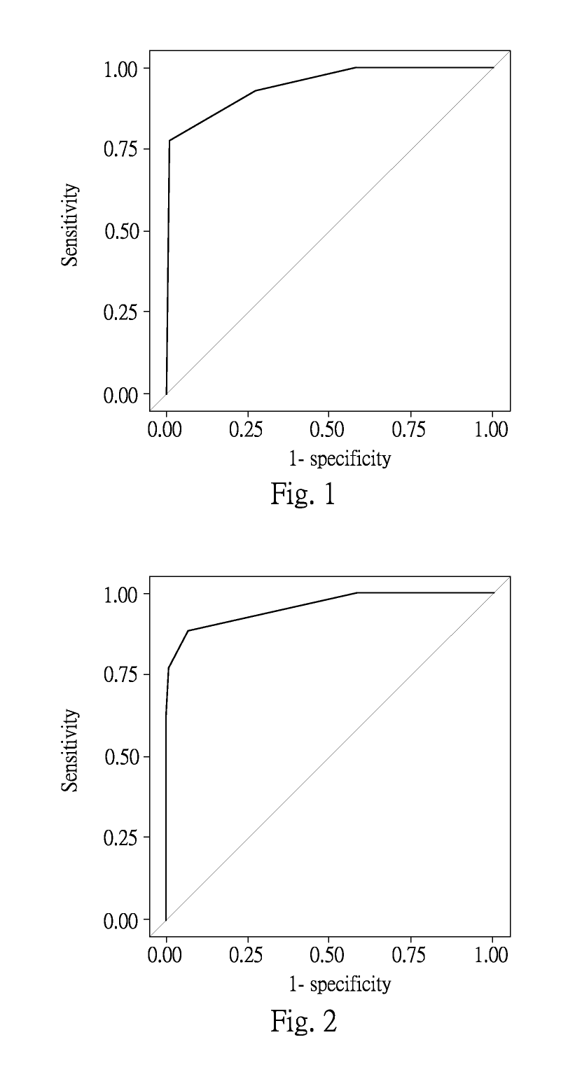

Obtaining the expression values of HIF1A and PGBD2 in each sample, all the collected data of HIF1A and PGBD2 were further analyzed by logistic regression analysis in SAS software as shown in table 6 and FIG. 1, wherein FIG. 1 is a ROC curve for predictive profile of detecting gastric cancer using HIF1A and PGBD2.

TABLE 6
The multivariate logistic regression result for HIF1A and PGBD2

| | | | 95% confidence | | |
|---|---|---|---|---|---|
| | | | interval | | |
| | | | Lower | Upper | |
| | β-estimate | Odds ratio | bound | bound | P value |
| Intercept | −1.1959 | | | | 0.0055 |
| HIF1A | | | | | |
| >0.93 | 4.7577 | 116.477 | 13.594 | 997.990 | <0.0001 |
| PGBD2 | | | | | |
| >2.34 | −5.7716 | 0.003 | <0.001 | 0.031 | <0.0001 |

The table 6 and FIG. 1 shows the predicted risk of gastric cancer in the examined patients according to the combinational biomarkers including HAF1A and PGBD2. According to table 6, it is known that a sample provider with HIF1A expression value higher than 0.93 shows 116.477 folds of risk of acquiring gastric cancer of with comparison of a sample provider with HIF1A expression value equal or less than 0.93 and a sample provider with PGBD2 expression value higher than 2.43 shows 0.003 fold of risk of acquiring gastric cancer with the comparison of a sample provider with HIF1A expression value equal or less than 2.43. In FIG. 1, the area under ROC curve is 0.9433 that suggests the accuracy of detecting gastric cancer by HIF1A and PGBD2 is 94.3%.

According to this example, while a sample is with HIF1A expression value higher than 0.93 and with PGBD2 expression value equal or less than 2.34, the sample provider is high-risk population for bearing gastric cancer with accuracy up to 94.3%. Therefore, the combination of HIF1A and PGBD2 can be provided to precisely diagnose gastric cancer.

### EXAMPLE 4

### A Combination of HIF1A, PGBD2 and FAM84B for Diagnosis Gastric Cancer

Obtaining the expression values of HIF1A, PGBD2 and FAM84B in each sample, all the expression values of HIF1A, PGBD2 and FAM84B were further analyzed by logistic regression analysis in SAS software as shown in table 7 and FIG. 2, wherein FIG. 2 is a ROC curve for predictive profile of detecting gastric cancer using HIF1A, PGBD2 and FAM84B.

TABLE 7
The multivariate logistic regression results for HIF1A, PGBD2
and FAM84B

| | | | 95% confidence | | |
|---|---|---|---|---|---|
| | | | interval | | |
| | | Point | Upper | Lower | |
| | β-estimate | estimate | bound | bound | P value |
| Intercept | −0.0695 | | | | 0.906 |
| aHIF1A | 4.5831 | 97.818 | 10.48 | 912.988 | <.0001 |
| aPGBD2 | −4.8025 | 0.008 | <0.001 | 0.085 | <.0001 |
| aFAM84B | −2.0703 | 0.126 | 0.026 | 0.622 | 0.011 |

The table 7 and FIG. 2 show the risk of acquiring gastric cancer in the sample provider upon examination of the expression values of three biomarkers including HIF1A, PGBD2 and FAM84B in the sample at the same time. According to table 7, it is known that a sample provider with HIF1A expression value higher than 0.93 shows 97.818 folds of risk of risk of acquiring gastric cancer with comparison of a sample provider with HIF1A expression value equal or less than 0.93; a sample provider with PGBD2 expression value higher than 2.43 shows 0.008 fold of risk of acquiring gastric cancer with the comparison of a sample provider with PGBD2 expression value equal or less than 2.43; and a sample provider with FAM84B expression value higher than 0.05 shows 0.126 fold of risk of acquiring gastric cancer with the comparison of a sample provider with FAM84B expression value equal or less than 0.05. In FIG. 2, the area under ROC curve is 0.9564 that suggests the accuracy in predicting gastric cancer by the combination of HIF1A, PGBD2 and FAM84B is 95.64%.

According to this example, while a sample is with HIF1A expression value higher than 0.93, with PGBD2 expression value equal or less than 2.34 and with FAM84B expression value equal or less than 0.05, the sample provider is high-risk population for bearing early gastric cancer with accuracy up to 95.64%. Therefore, the combination of HIF1A, PGBD2 and FAM84B can be provided to precisely diagnose gastric cancer.

### EXAMPLE 5

### A Combination of HIF1A, PGBD2 and CRIP2 for Diagnosis Gastric Cancer

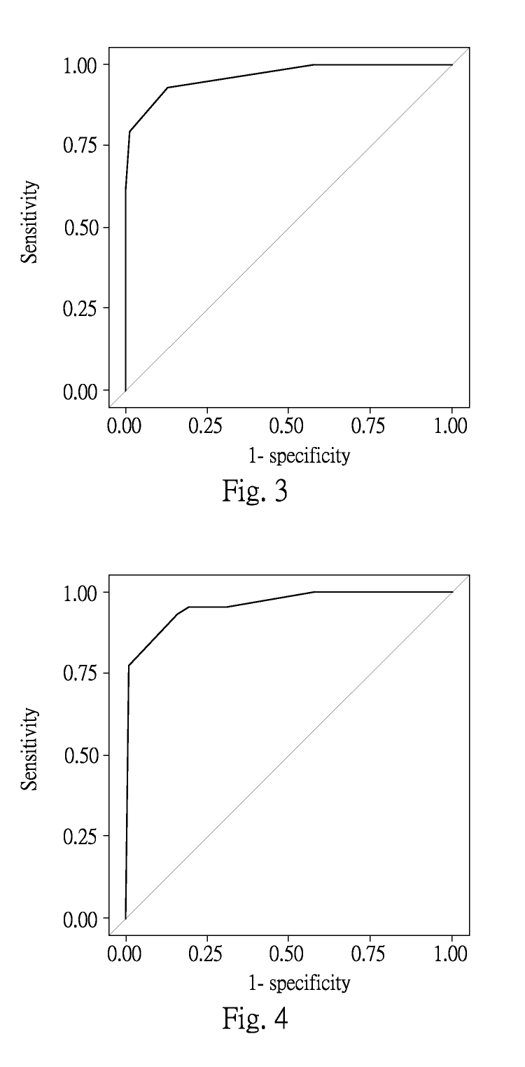

Obtaining the expression values of HIF1A, PGBD2 and CRIP2 in each sample, all the expression values of HIF1A, PGBD2 and CRIP2 were further analyzed by logistic regression analysis in SAS software as shown in table 8 and FIG. 3, wherein FIG. 3 is a ROC curve for predictive profile of detecting gastric cancer using HIF1A, PGBD2 and CRIP2.

TABLE 8
The multivariate logistic regression results for HIF1A, PGBD2
and FAM84B

| | | | 95% confidence | | |
|---|---|---|---|---|---|
| | | | interval | | |
| | | | Upper | Lower | |
| | β-estimate | Point-estimate | bound | bound | P value |
| Intercept | −0.3827 | | | | 0.4392 |
| aHIF1A | 5.5534 | 258.123 | 16.285 | >999.999 | <.0001 |
| aPGBD2 | −4.6314 | 0.01 | <0.001 | 0.105 | 0.0001 |
| aCRIP2 | −2.9795 | 0.051 | 0.005 | 0.489 | 0.0099 |

The table 8 and FIG. 3 show the risk of gastric cancer predicted by the expression values of three biomarkers including HIF1A, PGBD2 and CRIP2 in the sample at the same time. According to the table 8, it is known that a sample provider with HIF1A expression value higher than 0.93 shows 258.123 folds of risk of acquiring gastric cancer with comparison of a sample provider with HIF1A expression value equal or less than 0.93; a sample provider PGBD2 expression higher than 2.43 shows 0.01 fold of risk of acquiring gastric cancer with comparison of a sample provider with PGBD2 expression value equal or less than 0.243; and a sample provider with CRIP2 expression value higher than 2.73 shows 0.051 fold of risk of acquiring gastric cancer with comparison of a sample provider with PGBD2 expression value equal or less than 2.71. In FIG. 3, the area under ROC curve is 0.965 that suggests the accuracy in predicting gastric cancer by the combination of HIF1A, PGBD2 and CRIP2 is 96.5%.

According to this example, while a sample is with HIF1A expression value higher than 0.93, with PGBD2 expression value equal or less than 2.34 and with CRIP2 expression value equal or less than 2.73, the sample provider is high-risk population for bearing early gastric cancer with accuracy up to 95.64%. Therefore, the combination of HIF1A, PGBD2 and CRIP2 can be provided to precisely diagnose gastric cancer.

### EXAMPLE 6

### A Combination of HIF1A, PGBD2 and RPL15 for Diagnosis Gastric Cancer

Obtaining the expression values of HIF1A, PGBD2 and RPL15 in each sample, all the expression values of HIF1A, PGBD2 and RPL15 were further analyzed by logistic regression analysis in SAS software value as shown in table 9 and FIG. 4, wherein FIG. 4 is a ROC curve for predictive profile of detecting gastric cancer using HIF1A, PGBD2 and RPL15.

TABLE 9
The multivariate logistic regression results for HIF1A,
PGBD2 and RPL15

| | | | 95% confidence | | |
|---|---|---|---|---|---|
| | | | interval | | |
| | | | Upper | Lower | |
| | β-estimate | Point-estimate | bound | bound | P value |
| Intercept | −0.7792 | | | | 0.0916 |
| aHIF1A | 4.7916 | 120.5 | 12.922 | >999.999 | <.0001 |
| aPGBD2 | −4.8099 | 0.008 | <0.001 | 0.088 | <.0001 |
| aRPL15 | −1.8493 | 0.157 | 0.029 | 0.858 | 0.0325 |

Table 9 and FIG. 4 show the risk of acquiring gastric cancer calculated upon the expression values of three biomarkers including HIF1A, PGBD2 and RPL15 in the sample at the same time. According to table 9, it is known a sample provider with HIF1A expression value higher than 0.93 shows 120.5 folds of risk of acquiring gastric cancer with comparison of a sample provider with HIF1A expression value equal or less than 0.93; a sample provider with PGBD2 expression value higher than 2.43 shows 0.008 fold of risk of acquiring gastric cancer with comparison of a sample provider with PGBD2 expression value equal or less than 2.43; and a sample provider with RPL15 expression value higher than 2.14 shows 0.157 fold of risk of acquiring gastric cancer with comparison of a sample provider with RPL15 expression value equal or less than 2.14. In FIG. 4, the area under ROC curve is 0.9569 that suggests the accuracy in predicting gastric cancer by the combination of HIF1A, PGBD2 and RPL15 is 95.69%.

Based on this example, while a sample with HIF1A expression value higher than 0.93, with PGBD2 expression value equal or less than 2.34 and with RPL15 expression value equal or less than 2.14, the sample provider is high-risk population for bearing early gastric cancer with accuracy up to 95.69%. Therefore, the combination of HIF1A, PGBD2 and RPL15 can be provided to precisely diagnose gastric cancer.

### EXAMPLE 7

### A Combination of HIF1A, PGBD2 and DLG1 for Diagnosis Gastric Cancer

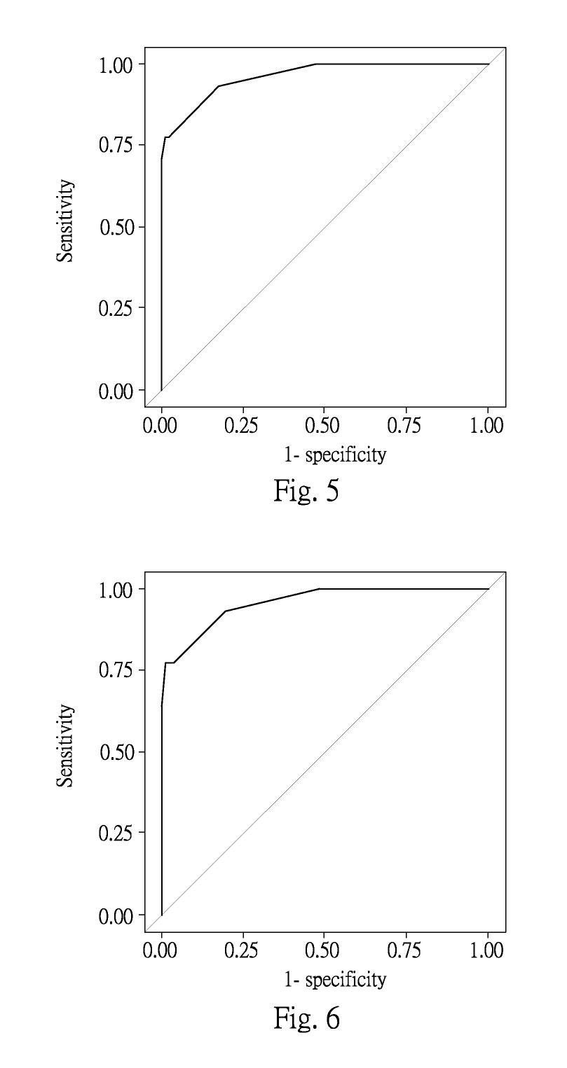

Obtaining the expression values of HIF1A, PGBD2 and DLG1 in each sample, all the expression values of HIF1A, PGBD2 and DLG1 were further analyzed by logistic regression analysis in SAS software as shown in table 10 and FIG. 5, wherein FIG. 5 is a ROC curve for predictive profile of detecting gastric cancer using HIF1A, PGBD2 and DLG1.

TABLE 10
The multivariate logistic regression results for HIF1A,
PGBD2 and DLG1

| | | | 95% confidence | | |
|---|---|---|---|---|---|
| | | | interval | | |
| | | Point- | Upper | Lower | |
| | β-estimate | estimate | bound | bound | P value |
| Intercept | −0.7002 | | | | 0.1298 |
| aHIF1A | 5.2883 | 197.998 | 14.381 | >999.999 | <.0001 |
| aPGBD2 | −4.1784 | 0.015 | 0.001 | 0.174 | 0.0007 |
| aDLG1 | −2.8557 | 0.058 | 0.005 | 0.625 | 0.0189 |

The table 10 and FIG. 5 show the risk of acquiring gastric cancer calculated upon the expression values of three biomarkers including HIF1A, PGBD2 and FAM84B in the sample at the same time. As known in table 10, a sample provider with HIF1A expression value higher than 0.93 shows 197.998 folds of risk of acquiring gastric cancer with comparison of a sample provider with HIF1A expression equal or less than 0.93; a sample provider with PGBD2 expression value higher than 2.43 shows 0.015 fold of risk of acquiring gastric cancer with comparison of a sample provider with PGBD2 expression value equal or less than 2.43; and a sample provider with DLG1 expression value higher than 0.96 show 0.058 fold of risk of acquiring gastric cancer with comparison of a sample provider with DLG1 expression value equal or less than 0.96. In FIG. 5, the area under ROC curve is 0.9621 that suggests the accuracy in predicting gastric cancer by HIF1A, PGBD2 and DLG1 is 96.21%.

According to this example, while a sample is with HIF1A expression value higher than 0.93, with PGBD2 expression value equal or less than 2.34 and with DLG1 expression value equal or less than 0.96, the sample provider is high-risk population for bearing early gastric cancer with accuracy up to 96.21%. Therefore, the combination of HIF1A, PGBD2 and DLG1 can be provided to precisely diagnose gastric cancer.

### EXAMPLE 8

### A Combination of HIF1A, PGBD2 and MAT2A for Diagnosis Gastric Cancer

Obtaining the expression values of HIF1A, PGBD2 and MAT2A in each sample, all the expression values of HIF1A, PGBD2 and MAT2A were further analyzed by logistic regression analysis in SAS software as shown in table 11 and FIG. 6, wherein FIG. 6 is a ROC curve for predictive profile of detecting gastric cancer using HIF1A, PGBD2 and MAT2A.

TABLE 11
The multivariate logistic regression results for HIF1A, PGBD2
and MAT2A

| | | | 95% confidence | | |
|---|---|---|---|---|---|
| | | | interval | | |
| | | Point- | Upper | Lower | |
| | β-estimate | estimate | bound | bound | P value |
| Intercept | −0.8062 | | | | 0.0779 |
| aHIF1A | 5.2369 | 188.085 | 16.492 | >999.999 | <.0001 |
| aPGBD2 | −4.8215 | 0.008 | <0.001 | 0.085 | <.0001 |
| aMAT2A | −2.0999 | 0.122 | 0.017 | 0.865 | 0.0353 |

The table 11 and FIG. 6 show the risk of acquiring gastric cancer calculated upon the expression values of three biomarkers including HIF1A, PGBD2 and MAT2A in the sample at the same time. As known in table 11, a sample provider with HIF1A expression value higher than 0.93 shows 188.085 folds of risk of acquiring gastric cancer with comparison of a sample provider with HIF1A expression value equal or less than 0.93; a sample provider with PGBD2 expression value higher than 2.43 shows 0.008 fold of risk of acquiring gastric cancer with comparison of a sample provider with PGBD2 expression value equal or less than 2.43; and a sample provider with MAT2A expression value higher than 1.08 shows 0.122 fold of risk of acquiring gastric cancer with comparison of a sample provider with PGBD2 expression value equal or less than 1.08. In FIG. 6, the area under ROC curve is 0.9578 that suggests the accuracy in predicting gastric cancer by HIF1A, PGBD2 and MAT2A is 95.78%.

According to this example, while a sample is with HIF1A expression value higher than 0.93, with PGBD2 expression value equal or less than 2.34 and with MAT2A expression value equal or less than 1.08, the sample provider is high-risk population for bearing early gastric cancer with accuracy up to 95.78%. Therefore, the combination of HIF1A, PGBD2 and MAT2A can be provided to precisely diagnose gastric cancer.

### EXAMPLE 9

### A Combination of HIF1A, PGBD2 and ID3 for Diagnosis Gastric Cancer

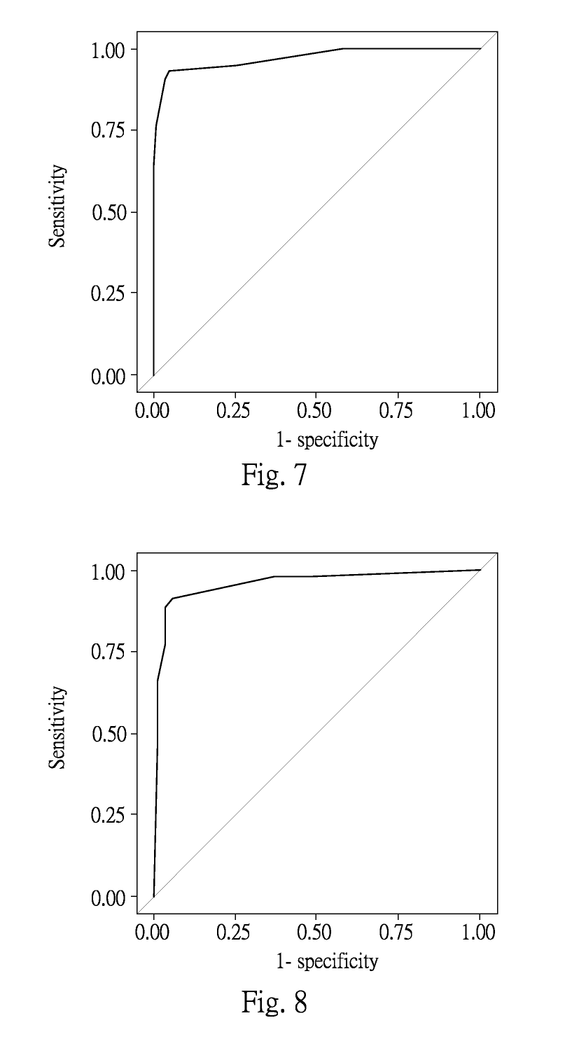

Obtaining the expression value of HIF1A, PGBD2 and ID3 in each sample, all the expression values of HIF1A, PGBD2 and ID3 were further analyzed by logistic regression analysis in SAS software as shown in table 12 and FIG. 7, wherein FIG. 7 is a ROC curve for predictive profile of detecting gastric cancer using HIF1A, PGBD2 and ID3.

TABLE 12
The multivariate logistic regression result for HIF1A, PGBD2
and MAT2A

| | | | 95% confidence | | |
|---|---|---|---|---|---|
| | | | interval | | |
| | | Point- | Upper | Lower | |
| | β-estimate | estimate | bound | bound | P value |
| Intercept | 0.8863 | | | | 0.211 |
| aHIF1A | 4.7674 | 117.618 | 9.764 | >999.999 | 0.0002 |
| aPGBD2 | −4.8198 | 0.008 | <0.001 | 0.095 | 0.0001 |
| aID3 | −3.6707 | 0.025 | 0.004 | 0.162 | 0.0001 |

The table 12 and FIG. 7 show the risk of acquiring gastric cancer that is calculated by the expression values of three biomarkers including HIF1A, PGBD2 and ID3 in the sample at the same time. According to table 12, it is known that a sample provider with HIF1A expression value higher than 0.93 shows 117.618 folds of risk of acquiring gastric cancer with comparison of a sample provider with HIF1A expression value equal or less than 0.93; a sample provider with PGBD2 expression value higher than 2.43 shows 0.008 fold of risk of acquiring gastric cancer with comparison of a sample provider with PGBD2 expression value equal or less than 2.43; and a sample provider with ID3 expression greater than 0.37 shows 0.025 fold of risk of acquiring gastric cancer with comparison of a sample provider with ID3 expression equal or less than 0.37. In FIG. 7, the area under ROC curve is 0.9715 that suggests the accuracy in predicting gastric cancer by HIF1A, PGBD2 and ID3 expression is 97.15%.

According to this example, while a sample is with HIF1A expression value higher than 0.93, with PGBD2 expression value equal or less than 2.34 and with ID3 expression value equal or less than 0.37, the sample provider is high-risk population for bearing early gastric cancer with accuracy up to 97.15%. Therefore, the combination of HIF1A, PGBD2 and ID3 can be provided to precisely diagnose gastric cancer.

### EXAMPLE 10

### A Combination of HIF1A, FAM84B and ID3 for Diagnosis Gastric Cancer

Obtaining the expression values of HIF1A, FAM84B and ID3 in each sample, all the expression values of HIF1A, FAM84B and ID3 were further analyzed by logistic regression analysis in SAS software as shown in table 13 and FIG. 8, wherein FIG. 8 is a ROC curve for predictive profile of detecting gastric cancer using HIF1A, FAM84B and ID3.

TABLE 13
The multivariate logistic regression results for HIF1A,
FAM84B and ID3

| | | | 95% confidence | | |
|---|---|---|---|---|---|
| | | | interval | | |
| | | Point- | Upper | Lower | |
| | β-estimate | estimate | bound | bound | P value |
| Intercept | 1.1852 | | | | 0.0961 |
| aHIF1A | 3.2358 | 25.428 | 4.313 | 149.898 | 0.0004 |
| aFAM84B | −2.3643 | 0.094 | 0.02 | 0.431 | 0.0024 |
| aID3 | −3.7307 | 0.024 | 0.005 | 0.125 | <.0001 |

The table 13 and FIG. 8 show the risk of acquiring gastric cancer that is calculated by the expression values of three biomarkers including HIF1A, FAM84B and ID3 in the sample at the same time. As shown in table 13, a sample provider with HIF1A expression value higher than 0.93 shows 25.428 folds of risk of acquiring gastric cancer with comparison of a sample provider with HIF1A expression value equal or less than 0.93; a sample provider with FAM84B expression value higher than 0.05 shows 0.094 fold of risk of acquiring gastric cancer with comparison of a sample provider with FAM84B expression value equal or less than 0.05; and a sample provider with ID3 expression value higher than 0.37 shows 0.024 fold of risk of acquiring gastric cancer with comparison of a sample provider with ID3 expression value equal or less than 0.37. In FIG. 6, the area under ROC curve is 0.9566 that suggests the accuracy in predicting gastric cancer by HIF1A, FAM84B and ID3 is 95.66%.

According to this example, while a sample is with HIF1A expression value higher than 0.93, with FAM84B expression value equal or less than 0.05 and with ID3 expression value equal or less than 0.37, the sample provider is high-risk population for bearing early gastric cancer with accuracy up to 95.66%. Therefore, the combination of HIF1A, FAM84B and ID3 can be provided to precisely diagnose gastric cancer.

### EXAMPLE 11

### A Combination of HIF1A, PGBD2, CRIP2 and DLG1 for Diagnosis Gastric Cancer

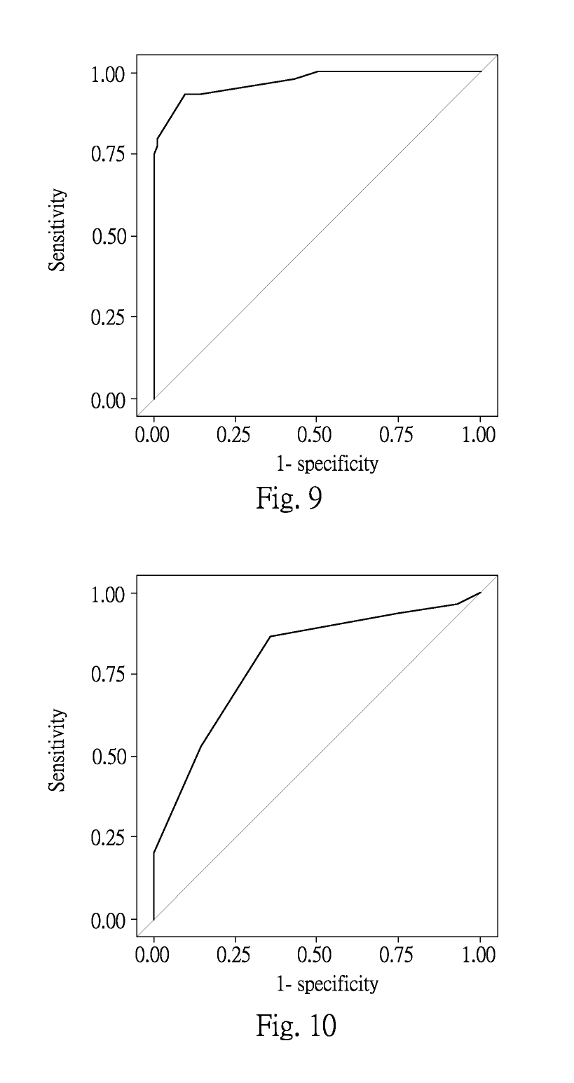

Obtaining the expression values of HIF1A, PGBD2, CRIP2 and DLG1 in each samples, all the expression values of HIF1A, PGBD2, CRIP2 and DLG1 were further analyzed by logistic regression analysis in SAS software as shown in table 14 and FIG. 9, wherein FIG. 9 is a ROC curve for predictive profile of detecting gastric cancer using HIF1A, PGBD2, CRIP2 and DLG1.

TABLE 14
The multivariate logistic regression result for the
combination of HIF1A, PGBD2, CRIP2 and DLG1

| | | | 95% confidence | | |
|---|---|---|---|---|---|
| | | | interval | | |
| | | | Upper | Lower | |
| | β-estimate | Point-estimate | bound | bound | P value |
| Intercept | −0.0746 | | | | 0.8868 |
| aHIF1A | 6.2732 | 530.191 | 18.1 | >999.999 | 0.0003 |
| aPGBD2 | −3.5172 | 0.03 | 0.002 | 0.354 | 0.0054 |
| aCRIP2 | −2.7683 | 0.063 | 0.006 | 0.683 | 0.023 |
| aDLG1 | −2.5523 | 0.078 | 0.006 | 0.989 | 0.049 |

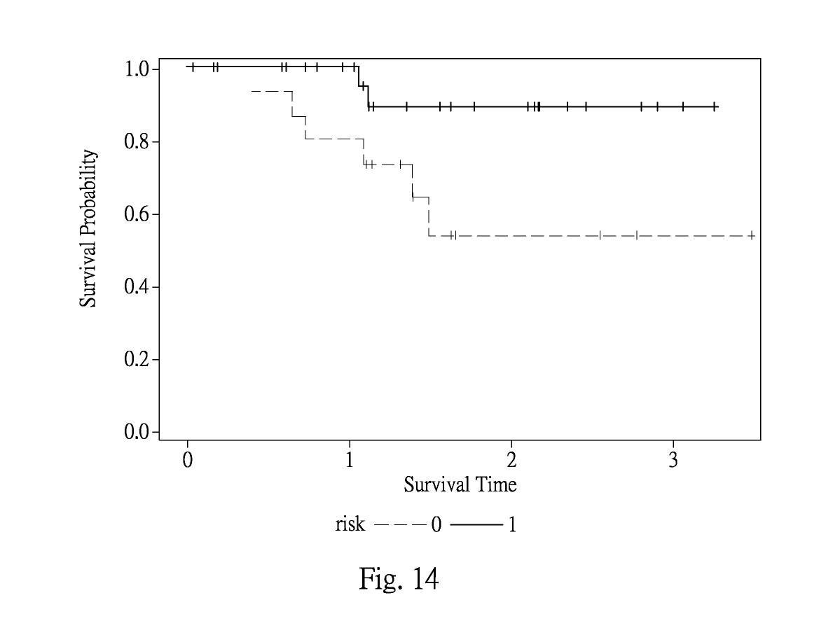

The table 14 and FIG. 9 show the risk of acquiring gastric cancer that is calculated by the expression values of the four biomarkers including HIF1A, PGBD2, CRIP2 and DLG1 in the sample at the same time. As known in FIG. 14, a sample provider with HIF1A expression value higher than 0.93 shows 530.191 folds of risk of acquiring gastric cancer with comparison of a sample provider with HIF1A expression value equal or less than 0.93; a sample provider with PGBD2 expression value higher than 2.43 shows 0.03 fold of risk of acquiring gastric cancer with comparison of a sample provider with PGBD2 expression value equal or less than 2.43; a sample provider with CRIP2 expression value higher than 2.73 shows 0.063 fold of risk of acquiring gastric cancer with comparison of a sample provider with CRIP2 expression value equal or less than 2.73; and a sample provider with DLG1 expression value higher than 0.96 show 0.074 fold of risk of acquiring gastric cancer with comparison of a sample provider with DLG1 expression value equal or less than 0.96. In FIG. 9, the area under ROC curve is 0.9688 that suggests the accuracy in predicting gastric cancer by HIF1A, PGBD2, CRIP2 and DLG1 is 96.88%.

According to this example, while a sample is with HIF1A expression value higher than 0.93, with PGBD2 expression value equal or less than 2.43, with CRIP2 expression value equal or less than 2.73 and with DLG1 expression value equal or less than 0.96, the sample provider is high-risk population for bearing early gastric cancer with accuracy up to 96.88%. Therefore, the combination of HIF1A, PGBD2, CRIP2 and DLG1 can be provided to precisely diagnose gastric cancer.

### EXAMPLE 12

### FAM84B or CRIP2 for Staging Gastric Cancer

In example 12, obtaining the expression values of the nine biomarkers in each gastric cancer sample, all the expression values of the nine biomarkers were further analyzed by Mann-Whitney U test and Student T test to determine whether the expression value of each biomarker exhibits the significant difference among different stages of gastric cancer. The statistic results are showed in the table 15, wherein P value with marker + was determined using ANOVA test and P value with marker * was determined using Kruskal-Wallis Test.

TABLE 15
The statistic results for the nine biomarkers of different stage of
gastric cancer

| | Gastric cancer stage | | | | |
|---|---|---|---|---|---|
| | Stage 1 | Stage 3 | Stage 3 | Stage 4 | P value |
| n | 8 | 6 | 11 | 19 | |
| HIF1A | | | | | |
| mean ± SD | 1.64 ± 0.73 | 1.93 ± 1.01 | 2.77 ± 1.86 | 1.55 ± 1.00 | 0.0787⁺ |
| median | 1.52 | 1.88 | 2.22 | 1.17 | 0.2407* |
| FAM84B | | | | | |
| mean ± SD | 0.09 ± 0.12 | 0.05 ± 0.04 | 0.04 ± 0.03 | 0.03 ± 0.03 | 0.1383⁺ |
| median | 0.06 | 0.05 | 0.03 | 0.03 | 0.5082* |
| CRIP2 | | | | | |
| mean ± SD | 4.65 ± 3.19 | 0.95 ± 0.99 | 1.75 ± 2.05 | 1.60 ± 1.40 | 0.0023⁺ |
| median | 4.30 | 0.58 | 1.48 | 1.15 | 0.0257* |
| GSN | | | | | |
| mean ± SD | 3.11 ± 1.21 | 3.79 ± 1.67 | 3.84 ± 2.23 | 3.50 ± 2.96 | 0.9196⁺ |
| median | 2.97 | 4.00 | 4.12 | 2.74 | 0.7793* |
| RPL15 | | | | | |
| mean ± SD | 2.07 ± 1.07 | 0.94 ± 0.53 | 1.39 ± 0.83 | 1.25 ± 0.70 | 0.0516⁺ |
| median | 1.98 | 0.95 | 1.24 | 1.09 | 0.1148* |
| DLG1 | | | | | |
| mean ± SD | 0.77 ± 0.47 | 0.40 ± 0.25 | 0.81 ± 0.49 | 0.49 ± 0.27 | 0.0515⁺ |
| median | 0.60 | 0.32 | 0.71 | 0.57 | 0.1678* |
| MAT2A | | | | | |
| mean ± SD | 0.83 ± 0.51 | 0.67 ± 0.40 | 0.79 ± 0.51 | 0.52 ± 0.28 | 0.2103⁺ |
| median | 0.64 | 0.52 | 0.62 | 0.50 | 0.3253* |
| PGBD2 | | | | | |
| mean ± SD | 1.22 ± 0.86 | 1.31 ± 0.61 | 1.61 ± 0.85 | 1.19 ± 0.65 | 0.5031⁺ |
| median | 0.86 | 1.45 | 1.47 | 1.09 | 0.4153* |
| ID3 | | | | | |
| mean ± SD | 0.36 ± 0.37 | 0.25 ± 0.15 | 0.31 ± 0.20 | 0.24 ± 0.21 | 0.5888⁺ |
| median | 0.24 | 0.22 | 0.24 | 0.19 | 0.5930* |

In addition, the samples were divided into early stage (Stage I & stage II) and late stage (Stage III & stage IV) gastric cancers for the further analysis by Mann-Whitney U test and Student T test to determine whether the expression value of each biomarker exhibits the significant difference among gastric cancer in different stages. The statistic results are showed in table 16, wherein P value with marker ++ was determined using chi-square test; P value with marker # was determined using fisher's exact test; P value with marker * was determined using Wilcoxon rank sum test; and P value with marker +was determined using Student t-test.

TABLE 16
The statistic results for the nine biomarkers in early and late gastric cancers

| | Gastric cancer stage | | Wilcoxon rank | |
|---|---|---|---|---|
| | Early stage | Late stage | sum test | P value |
| n | 14 | 30 | | |
| Age | | | | |
| mean ± SD | 60.38 ± 6.17 | 58.59 ± 5.76 | | 0.3541⁺ |
| Sex | | | | |
| Female | 4 (28.57%) | 15 (50.00%) | | |
| Male | 10 (71.43%) | 15 (50.00%) | | 0.1814^{‡} |
| Death | | | | |
| No | 14 (100.00%) | 22 (73.33%) | | |
| Yes | 0 (0.00) | 8 (26.67%) | | 0.0410⁺ |
| Tracing interval | | | | |
| (Days) | | | | |
| mean ± SD | 643.07 ± 373.44 | 494.3 ± 296.01 | | 0.1608⁺ |
| Recurrence | | | | |
| No | 14 (100.00%) | 23 (76.67%) | | |
| Yes | 0 (0.00) | 7 (23.33%) | | 0.0783^{#} |
| Recurrence | | | | |
| interval (Days) | | | | |
| mean ± SD | — | 224.14 ± 123.70 | | — |
| Lymph node | | | | |
| metastasis | | | | |
| No | 11 (78.57%) | 2 (6.67%) | | |
| Yes | 3 (21.43%) | 28 (93.33%) | | <0.0001^{#} |
| HIF1A | | | | |
| mean ± SD | 1.77 ± 0.84 | 2.00 ± 1.47 | | 0.5135⁺ |
| median | 1.69 | 1.66 | 319.5 | 0.9097* |
| FAM84B | | | | |
| mean ± SD | 0.07 ± 0.09 | 0.03 ± 0.03 | | 0.1671⁺ |
| median | 0.05 | 0.03 | 373.5 | 0.1403* |
| CRIP2 | | | | |
| mean ± SD | 3.06 ± 3.07 | 1.65 ± 1.63 | | 0.1258⁺ |
| median | 2.12 | 1.28 | 360.0 | 0.2568* |
| GSN | | | | |
| mean ± SD | 3.40 ± 1.41 | 3.62 ± 2.68 | | 0.7200⁺ |
| median | 3.06 | 3.23 | 332.0 | 0.6684* |
| RPL15 | | | | |
| mean ± SD | 1.58 ± 1.03 | 1.30 ± 0.74 | | 0.3029⁺ |
| median | 1.36 | 1.11 | 348.5 | 0.3985* |
| DLG1 | | | | |
| mean ± SD | 0.61 ± 0.42 | 0.61 ± 0.39 | | 0.9936⁺ |
| median | 0.51 | 0.59 | 305.5 | 0.8108* |
| MAT2A | | | | |
| mean ± SD | 0.76 ± 0.46 | 0.62 ± 0.40 | | 0.3111⁺ |
| median | 0.62 | 0.54 | 348.5 | 0.3985* |
| PGBD2 | | | | |
| mean ± SD | 1.26 ± 0.73 | 1.35 ± 0.75 | | 0.7161⁺ |
| median | 0.98 | 1.26 | 296.0 | 0.6320* |
| ID3 | | | | |
| mean ± SD | 0.32 ± 0.29 | 0.26 ± 0.21 | | 0.4841⁺ |
| median | 0.22 | 0.21 | 330.5 | 0.6958* |

Furthermore, the expression values of the nine biomarkers were respectively analyzed by logistic regression analysis in SAS software as shown in table 17.

TABLE 17
The univariate logistic regression results for the nine biomarkers

| | | | 95% confidence | | | | |
|---|---|---|---|---|---|---|---|
| | | | interval | | | | |
| | | Odds | Lower | Upper | | | Cut-off |
| | β-estimate | ratio | bound | bound | P value | AUC | value |
| HIF1A | 0.1482 | 1.160 | 0.685 | 1.964 | 0.5811 | 49.3% | 2.16 |
| FAM84B | −15.1169 | <0.001 | <0.001 | 78.960 | 0.1284 | 63.9% | 0.05 |
| CRIP2 | −0.2718 | 0.762 | 0.569 | 1.021 | 0.0686 | 60.1% | 1.92 |
| GSN | 0.0435 | 1.044 | 0.784 | 1.391 | 0.7662 | 47.6% | 3.15 |
| RPL15 | −0.3943 | 0.674 | 0.320 | 1.420 | 0.2997 | 58.3% | 1.12 |
| DLG1 | 0.00690 | 1.007 | 0.198 | 5.132 | 0.9934 | 50.7% | 0.52 |
| MAT2A | −0.7878 | 0.455 | 0.101 | 2.057 | 0.3063 | 57.9% | 0.60 |
| PGBD2 | 0.1689 | 1.184 | 0.488 | 2.873 | 0.7088 | 54.9% | 0.96 |
| ID3 | −0.9563 | 0.384 | 0.028 | 5.352 | 0.4767 | 53.7% | 0.27 |

According to the results shown in table 17, each biomarker was divided into two groups according to its cut-off value for the logistic regression analysis in SAS software. The analyzed results are showed in table 18, wherein P value* was determined using chi-square test or fisher's exact test; P value** was determined using chi-square test or Chi-square test with Yate's correction.

TABLE 18
The univariate logistic regression results for the two
groups of each biomarker divided by its cut off value

| | | Early gastric | Late gastric | | Odds ratio | | | |
|---|---|---|---|---|---|---|---|---|
| | Cut-off | cancer n = 14 | cancer n = 30 | | (95% confidence | | P | P |
| | value | Cases (%) | Cases (%) | | interval) | AUC | value* | value** |
| HIF1A | ≦2.16 | 10 (71.43) | 19 | (63.33) | 1 | | | |
| | >2.16 | 4 (28.57) | 11 | (36.67) | 1.4474 (0.3652, | 54.0% | 0.7384 | 0.8523 |
| | | | | | 5.7355) | | | |
| FAM84B | ≦0.05 | 7 (50.00) | 25 | (83.33) | 1 | | | |
| | >0.05 | 7 (50.00) | 5 | (16.67) | 0.2000 (0.0483, | 66.7% | 0.0208 | 0.0513 |
| | | | | | 0.8283) | | | |
| CRIP2 | ≦1.92 | 6 (42.86) | 23 | (76.67) | 1 | | | |
| | >1.92 | 8 (57.14) | 7 | (23.33) | 0.2283 (0.0589, | 66.9% | 0.0420 | 0.0626 |
| | | | | | 0.8850) | | | |
| GSN | ≦3.15 | 8 (57.14) | 14 | (46.67) | 1 | | | |
| | >3.15 | 6 (42.86) | 16 | (53.33) | 1.5238 (0.4243, | 55.2% | 0.5174 | 0.7462 |
| | | | | | 5.4731) | | | |
| RPL15 | ≦1.12 | 4 (28.57) | 15 | (50.00) | 1 | | | |
| | >1.12 | 10 (71.43) | 15 | (50.00) | 0.4000 (0.1024, | 60.7% | 0.1814 | 0.3126 |
| | | | | | 1.5625) | | | |
| DLG1 | ≦0.52 | 7 (50.00) | 12 | (40.00) | 1 | | | |
| | >0.52 | 7 (50.00) | 18 | (60.00) | 1.5000 (0.4182, | 55.0% | 0.5328 | 0.7665 |
| | | | | | 5.3796) | | | |
| MAT2A | ≦0.60 | 6 (42.86) | 17 | (56.67) | 1 | | | |
| | >0.60 | 8 (57.14) | 13 | (43.33) | 0.5735 (0.1592, | 56.9% | 0.3930 | 0.5960 |
| | | | | | 2.0656) | | | |
| PGBD2 | ≦0.96 | 7 (50.00) | 11 | (36.67) | 1 | | | |
| | >0.96 | 7 (50.00) | 19 | (63.33) | 1.7273 (0.4783, | 56.7% | 0.4021 | 0.6110 |
| | | | | | 6.2380) | | | |
| ID3 | ≦0.27 | 7 (50.00) | 21 | (70.00) | 1 | | | |
| | >0.27 | 7 (50.00) | 9 | (30.00) | 0.4286 (0.1160, | 60.0% | 0.1990 | 0.3431 |
| | | | | | 1.5830) | | | |

As shown in the table 18, the accuracy for the prediction of late gastric cancer by FAM84B is 66.7% and by CRIP2 is 66.9%. In detail, it is known that a sample provider with the expression value of FAM84B higher than 0.05 shows 0.2 fold of risk of acquiring late gastric cancer with comparison of a sample provider with the expression value of FAM84B equal or less than 0.05 and a sample provider with the expression value of CRIP2 higher than 1.92 shows 0.02283 fold of risk of acquiring late gastric cancer with comparison of a sample provider with the expression value of CRIP2 equal or less than 1.92. According to this example, it suggests that the expression value of FAM84B or CRIP2 is significantly associated with staging gastric cancer.

### EXAMPLE 13

### A Combination of CRIP2, DLG1 and MAT2A for Staging Gastric Cancer

Obtaining the expression values of the biomarkers including CRIP2, DLG1 and MAT2A in each sample, all the expression values of CRIP2, DLG1 and MAT2A were analyzed by logistic regression in SAS software as shown in table 19 and FIG. 10, wherein FIG. 10 is a ROC curve for predictive profile of detecting gastric cancer using CRIP2, DLG1 and MAT2A.

TABLE 19
The multivariate logistic regression
results for the CRIP2, DLG1 and MAT2A

| | | | 95% confidence | | | |
|---|---|---|---|---|---|---|
| | | | interval | | | |
| | | Odds | Lower | Upper | | |
| | Estimate β | ratio | bound | bound | P value | AUC |
| Intercept | 1.3964 | | | | 0.0248 | 79.8% |
| CRIP2 | | | | | | |
| >1.92 | −2.3503 | 0.095 | 0.018 | 0.501 | 0.0055 | |
| DLG1 | | | | | | |
| >0.52 | 2.8856 | 17.915 | 1.466 | 218.891 | 0.0238 | |
| MAT2A | | | | | | |
| >0.60 | −2.5863 | 0.075 | 0.007 | 0.814 | 0.0332 | |

As shown in table 19 and FIG. 10, a sample provider with CRIP2 expression value higher than 1.92 is 0.018 fold of risk of acquiring late gastric cancer with comparison of a sample provider with CRIP2 expression value equal or less than 1.92; a sample provider with DLG1 expression value higher than 0.52 shows 17.915 folds of risk of acquiring late gastric cancer with comparison of a sample provider with DLG1 expression value equal or less than 0.52; and a sample provider with MAT2A expression value higher than 0.60 shows 0.075 fold of risk of acquiring late gastric cancer with comparison of a sample provider with MAT2A expression value equal or less than 0.96. In FIG. 10, the area under ROC curve is 0.7976 that suggests the accuracy in predicting late gastric cancer by CRIP2, DLG1 and MAT2A is 79.76%.

According to this example, while a sample is with CRIP2 expression value equal or less than 1.92, with DLG1 expression value higher than 0.52 and with MAT2A expression value equal or less than 0.6, the sample provider has high risk for acquiring late gastric cancer with accuracy up to 79.76%. Therefore, the combination of CRIP2, DLG1 and MAT2A can be provided to staging gastric cancer.

### EXAMPLE 14

### A Combination of FAM84B, CRIP2, DLG1 and MAT2A for Staging Gastric Cancer

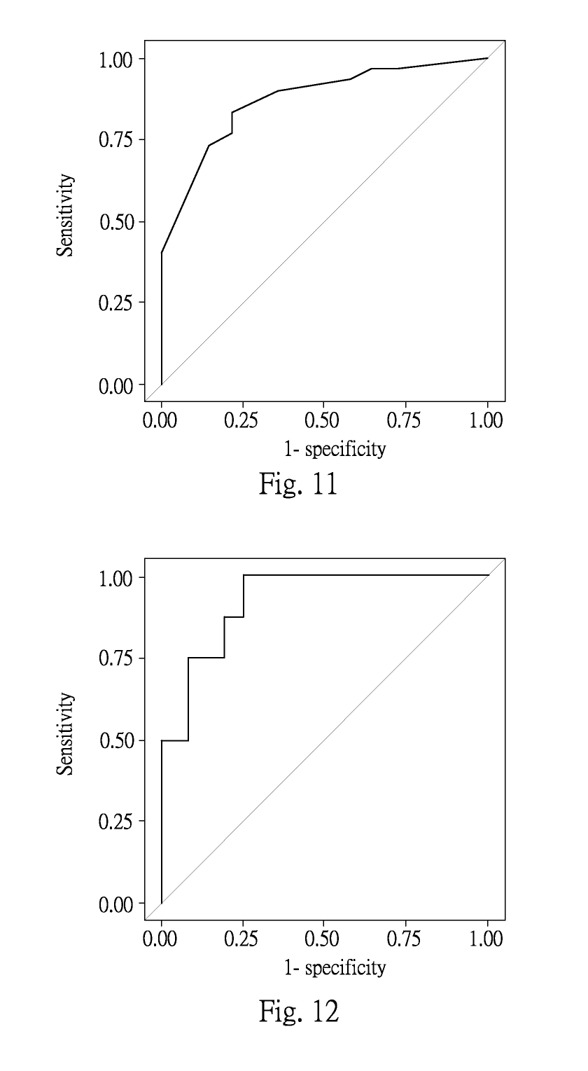

Obtaining the expression values of FAM84B, CRIP2, DLG1 and MAT2A in each sample, all the expression values of FAM84B, CRIP2, DLG1 and MAT2A were analyzed by logistic regression in SAS software as shown in table 20 and FIG. 11, wherein FIG. 9 is a ROC curve for predictive profile of detecting gastric cancer using FAM84B, CRIP2, DLG1 and MAT2A.

TABLE 20
The multivariate logistic regression results
for FAM84B, CRIP2, DLG1 and MAT2A

| | | | 95% confidence | | | |
|---|---|---|---|---|---|---|
| | | | interval | | | |
| | | Odds | Lower | Upper | | |
| | β-estimate | ratio | bound | bound | P value | AUC |
| Intercept | 1.6203 | | | | 0.0178 | 87.3% |
| FAM84B | | | | | | |
| >0.05 | −2.8833 | 0.056 | 0.005 | 0.675 | 0.0232 | |
| CRIP2 | | | | | | |
| >1.92 | −2.3409 | 0.096 | 0.015 | 0.619 | 0.0137 | |
| DLG1 | | | | | | |
| >0.52 | 4.6853 | 108.338 | 3.690 | >999.999 | 0.0066 | |
| MAT2A | | | | | | |
| >0.60 | −2.8497 | 0.058 | 0.004 | 0.763 | 0.0304 | |

The table 20 and FIG. 11 show that a sample provider with FAM84B expression value higher than 0.05 show 0.056 fold of risk of acquiring late gastric cancer with comparison of a sample provider with FAM84B expression value equal or less than 0.05; a sample provider with CRIP2 expression value higher than 1.92 shows 0.096 fold of risk of acquiring late gastric cancer with comparison of a sample provider with CRIP2 expression value equal or less than 1.92; a sample provider with DLG1 expression value higher than 0.52 shows 108.338 folds of risk of acquiring late gastric cancer with comparison of a sample provider with DLG1 expression equal or less than 0.52; and a sample provider with MAT2A expression value higher than 0.60 shows 0.058 fold of risk of acquiring late gastric cancer with comparison of a sample provider with MAT2A expression value equal or less than 0.60. In FIG. 10, the area under ROC curve is 0.8726 that suggests the accuracy in predicting late gastric cancer by FAM84B, CRIP2, DLG1 and MAT2A is 87.26%.

The statistic results in table 20 and FIG. 11 suggest that a sample is with FAM84B expression value equal or less than 0.05, with CRIP2 expression value equal or less than 1.92, with DLG1 expression value higher than 0.52 and with MAT2A expression value equal or less than 0.6, the sample provider has high risk for acquiring late gastric cancer with accuracy up to 87.26%. Therefore, the combination of FAM84B, CRIP2, DLG1 and MAT2A can be provided to staging gastric cancer.

Taken together, the statistic results shown in table 18 to table 20 suggest that a sample provider with the increased DLG1 expression value and decreased FAM84B, CRIP2 and MAT2A expression values is the high-risk population in acquiring late gastric cancer.

### EXAMPLE 15

### CRIP2 or RPL15 for Predicting Lymph Node Metastasis

In example 15, obtaining the expression values of the nine biomarkers from each gastric cancer sample, all the collected date of the nine biomarkers were respectively analyzed by Mann-Whitney U test and Student T test to determine whether the expression value of each biomarker exhibits the significant difference in the patients with and without lymph node metastasis. The statistic results are shown in table 21, wherein P value with marker ++ was determined using chi-square test; P value with marker # was determined using fisher's exact test; P value with marker * was determined using Wilcoxon rank sum test; and P value with marker +was determined using Student t-test.

TABLE 21
The statistic results for the nine biomarkers of the patients with or without
lymph node metastasis

| | | Lymph node | | Wilcoxon | |
|---|---|---|---|---|---|
| | | Non-metastasis | Metastasis | rank sum | P value |
| | Numbers | 13 (29.55%) | 31 (70.45%) | | |
| Age | mean ± SD | 60.28 ± 6.15 | 58.69 ± 5.80 | | 0.4187⁺ |
| Sex | Female | 3 (23.08%) | 16 (51.61%) | | 0.0812^{‡} |
| | Male | 10 (76.92%) | 15 (48.39%) | | |
| Stage | 1 | 8 (61.54%) | 0 (0) | | |
| | 2 | 3 (23.08%) | 3 (9.68%) | | |
| | 3 | 1 (7.69%) | 10 (32.26%) | | |
| | 4 | 1 (7.69%) | 18 (58.06%) | | |
| Death | No | 13 (100%) | 23 (74.19%) | | 0.0820^{#} |
| | Yes | 0 (0) | 8 (25.81%) | | |
| Tracing | mean ± SD | 559.69 ± 360.42 | 534.06 ± 316.25 | | 0.8150⁺ |
| interval | | | | | |
| Recurrence | mean ± SD | | 224.14 ± 123.70 | | |
| interval | | | | | |
| Recurrence | No | 13 (100%) | 24 (77.42%) | | 0.0857^{#} |
| | Yes | 0 (0) | 7 (22.58%) | | |
| HIF1A | mean ± SD | 1.9 ± 0.93 | 1.93 ± 1.44 | | 0.9432⁺ |
| | median | 1.83 | 1.55 | 316.5 | 0.5369* |
| FAM84B | mean ± SD | 0.07 ± 0.09 | 0.04 ± 0.03 | | 0.1785⁺ |
| | median | 0.04 | 0.03 | 346.0 | 0.1686* |
| CRIP2 | mean ± SD | 3.71 ± 2.83 | 1.43 ± 1.58 | | 0.0149⁺ |
| | median | 2.73 | 1.09 | 412.0 | 0.0021* |
| GSN | mean ± SD | 3.09 ± 1.52 | 3.75 ± 2.6 | | 0.3958⁺ |
| | median | 2.86 | 3.31 | 276.0 | 0.6712* |
| RPL15 | mean ± SD | 1.72 ± 0.96 | 1.25 ± 0.76 | | 0.0932⁺ |
| | median | 1.44 | 1.1 | 361.5 | 0.0759* |
| DLG1 | mean ± SD | 0.67 ± 0.39 | 0.58 ± 0.4 | | 0.5196⁺ |
| | median | 0.6 | 0.54 | 317.5 | 0.5201* |
| MAT2A | mean ± SD | 0.74 ± 0.45 | 0.64 ± 0.41 | | 0.4498⁺ |
| | median | 0.56 | 0.52 | 320.5 | 0.4713* |
| PGBD2 | mean ± SD | 1.35 ± 0.75 | 1.3 ± 0.74 | | 0.8365⁺ |
| | median | 1.04 | 1.27 | 298.0 | 0.8875* |
| ID3 | mean ± SD | 0.32 ± 0.3 | 0.26 ± 0.21 | | 0.5010⁺ |
| | median | 0.16 | 0.21 | 298.5 | 0.8772* |

The expression values of the nine biomarkers were further analyzed using logistic regression analysis in SAS software as shown in table 22.

TABLE 22
The univariate logistic regression results for the nine biomarkers

| | | | 95% confidence | | | | |
|---|---|---|---|---|---|---|---|
| | | | interval | | | | |
| | | Odds | Lower | Upper | | | Cut-off |
| | Estimate β | ratio | bound | bound | P value | AUC | value |
| HIF1A | 0.0190 | 1.019 | 0.613 | 1.695 | 0.9415 | 44.0% | 2.91 |
| FAM84B | −14.9346 | <0.001 | <0.001 | 83.965 | 0.1306 | 63.3% | 0.06 |
| CRIP2 | −0.4792 | 0.619 | 0.431 | 0.891 | 0.0097 | 79.7% | 1.92 |
| GSN | 0.1469 | 1.158 | 0.827 | 1.621 | 0.3918 | 54.1% | 3.15 |
| RPL15 | −0.6447 | 0.525 | 0.242 | 1.140 | 0.1032 | 67.1% | 1.24 |
| DLG1 | −0.5439 | 0.580 | 0.115 | 2.941 | 0.5112 | 56.2% | 0.39 |
| MAT2A | −0.6000 | 0.549 | 0.119 | 2.529 | 0.4415 | 56.9% | 0.52 |
| PGBD2 | −0.0958 | 0.909 | 0.375 | 2.201 | 0.8319 | 51.4% | 1.88 |
| ID3 | −0.9325 | 0.394 | 0.027 | 5.672 | 0.4933 | 51.5% | 0.28 |

The statistic results shown in table 22 reveal that the expression value of CRIP2 is significantly correlated with the lymph node metastasis in the gastric cancer patients. Furthermore, the accuracy of the prediction of lymph node metastasis in gastric cancer patients relies on CRIP2 expression value is 79.7%.

Moreover, each of the nine biomarkers was divided into two groups by its cut-off value for the further examination by logistic regression analysis in SAS software. The statistic results are shown in table 23, wherein the p value was determined using chi-square or fisher's exact test.

TABLE 23
The univariate logistic regression results for the two
groups of each biomarker divided by its cut off value

| | | Non-lymph node | | Lymph node | | | | |
|---|---|---|---|---|---|---|---|---|
| | | metastasis | | metastasis | | Odds ratio | | |
| | Cut-off | n = 13 | | n = 31 | | (95% confidence | P | |
| | value | Case (%) | | Case (%) | | interval) | value | AUC |
| HIF1A | ≦2.91 | 11 | (84.62) | 24 | (77.42) | 1 | | |
| | >2.91 | 2 | (15.38) | 7 | (22.58) | 1.60 (0.29, | 0.7030 | 53.6% |
| | | | | | | 9.01) | | |
| FAM84B | ≦0.06 | 8 | (61.54) | 27 | (87.1) | 1 | | |
| | >0.06 | 5 | (38.46) | 4 | (12.9) | 0.24 (0.05, | 0.0976 | 62.8% |
| | | | | | | 1.10) | | |
| CRIP2 | ≦1.92 | 3 | (23.08) | 26 | (83.87) | 1 | | |
| | >1.92 | 10 | (76.92) | 5 | (16.13) | 0.06 (0.01, | 0.0005 | 80.4% |
| | | | | | | 0.29) | | |
| GSN | ≦3.15 | 8 | (61.54) | 14 | (45.16) | 1 | | |
| | >3.15 | 5 | (38.46) | 17 | (54.84) | 1.94 (0.52, | 0.3250 | 58.2% |
| | | | | | | 7.29) | | |
| RPL15 | ≦1.24 | 4 | (30.77) | 20 | (64.52) | 1 | | |
| | >1.24 | 9 | (69.23) | 11 | (35.48) | 0.24 (0.06, | 0.0468 | 66.9% |
| | | | | | | 0.98) | | |
| DLG1 | ≦0.39 | 3 | (23.08) | 12 | (38.71) | 1 | | |
| | >0.39 | 10 | (76.92) | 19 | (61.29) | 0.48 (0.11, | 0.4884 | 57.8% |
| | | | | | | 2.08) | | |
| MAT2A | ≦0.52 | 4 | (30.77) | 16 | (51.61) | 1 | | |
| | >0.52 | 9 | (69.23) | 15 | (48.39) | 0.42 (0.11, | 0.2112 | 60.4% |
| | | | | | | 1.64) | | |
| PGBD2 | ≦1.88 | 9 | (69.23) | 25 | (80.65) | 1 | | |
| | >1.88 | 4 | (30.77) | 6 | (19.35) | 0.54 (0.12, | 0.4489 | 55.7% |
| | | | | | | 2.36) | | |
| ID3 | ≦0.28 | 7 | (53.85) | 22 | (70.97) | 1 | | |
| | >0.28 | 6 | (46.15) | 9 | (29.03) | 0.48 (0.13, | 0.3129 | 58.6% |
| | | | | | | 1.82) | | |

Table 23 reveals that the expression value of CRIP2 or RPL15 is significantly correlated with the lymph node metastasis in the gastric cancer patients. As shown in table 23, a sample provider with CRIP2 expression value higher than 1.92 shows 0.06 fold of risk of lymph node metastasis with comparison of a sample provider with CRIP2 expression value equal or less than 1.92 and a sample provider with RPL15 expression value higher than 1.24 shows 0.24 fold of risk of lymph node metastasis with comparison of a sample provider with RPL15 expression value equal or less than 1.24. Furthermore, the accuracy in predicting the lymph node metastasis relies on CRIP2 is 80.4% and on RPL15 is 66.9%.

Collectively, the results shown in tables 22 and 23 reveal that the patients with decreased expression value of CRIP2 or RPL15 are high-risk population in lymph node metastasis.

### EXAMPLE 16

### The Biomarkers for Predicting Survival Rate

In example 16, obtaining the expression values of the nine biomarkers in each gastric cancer sample, all the expression values of the nine biomarkers were analyzed by Mann-Whitney U test and Student T test to determine whether the expression value of each biomarker is significantly correlated with the post-survival rate. The statistic results are shown in table 24, herein, P value with marker +was determined using Student-t test; P value with marker # was determined using fisher's exact test; P value with marker * was determined using Wilcoxon rank sum test; and P value with marker + was determined using Student t-test.

TABLE 23
The statistic results of prognosis for the nine biomarkers

| | | | Wilcoxon rank | |
|---|---|---|---|---|
| | Survival | Death | sum | P value |
| n | 36 | 8 | | |
| Age | | | | |
| mean ± SD | 59.56 ± 6.11 | 57.37 ± 4.61 | | 0.3466⁺ |
| median | 58.07 | 56.27 | 147.0 | 0.3153* |
| Sex | | | | |
| Female | 17 (47.22%) | 2 (25.00%) | | 0.4329^{#} |
| Male | 19 (52.78%) | 6 (75.00%) | | |
| Stage | | | | |
| 1 | 8 (22.22%) | 0 (0.00) | | |
| 2 | 6 (16.67%) | 0 (0.00) | | |
| 3 | 8 (22.22%) | 3 (37.50%) | | |
| 4 | 14 (38.89%) | 5 (62.50%) | | |
| Tracing interval | | | | |
| (Days) | | | | |
| mean ± SD | 581.67 ± 343.02 | 361.50 ± 136.17 | | 0.0063⁺ |
| median | 540.50 | 392.00 | 119.5 | 0.0656* |
| Recurrence (n = 44) | | | | |
| No | 31 (86.11%) | 6 (75.00%) | | 0.5934^{#} |
| Yes | 5 (13.89%) | 2 (25.00%) | | |
| Recurrence | | | | |
| interval | | | | |
| (Days) | | | | |
| mean ± SD | 273.40 ± 110.50 | 101.00 ± 22.63 | | 0.0928⁺ |
| median | 286.00 | 101.00 | 3.0 | 0.0528* |
| Lymph node | | | | |
| metastasis | | | | |
| No | 13 (36.11%) | 0 (0.00) | | 0.0820^{#} |
| Yes | 23 (63.89%) | 8 (100.00%) | | |
| HIF1A | | | | |
| mean ± SD | 1.96 ± 1.25 | 1.75 ± 1.56 | | 0.6848⁺ |
| median | 1.66 | 1.53 | 165.0 | 0.6480* |
| FAM84B | | | | |
| mean ± SD | 0.05 ± 0.06 | 0.02 ± 0.02 | | 0.0235⁺ |
| median | 0.04 | 0.02 | 128.0 | 0.1134* |
| CRIP2 | | | | |
| mean ± SD | 2.39 ± 2.37 | 0.82 ± 0.94 | | 0.0049⁺ |
| median | 1.51 | 0.36 | 101.0 | 0.0162* |
| GSN | | | | |
| mean ± SD | 3.45 ± 1.64 | 4.03 ± 4.45 | | 0.7248⁺ |
| median | 3.23 | 1.77 | 148.0 | 0.3302* |
| RPL15 | | | | |
| mean ± SD | 1.45 ± 0.89 | 1.11 ± 0.57 | | 0.3037⁺ |
| median | 1.23 | 0.98 | 142.5 | 0.2538* |
| DLG1 | | | | |
| mean ± SD | 0.62 ± 0.40 | 0.56 ± 0.37 | | 0.6750⁺ |
| median | 0.54 | 0.60 | 174.0 | 0.8551* |
| MAT2A | | | | |
| mean ± SD | 0.66 ± 0.40 | 0.70 ± 0.60 | | 0.7869⁺ |
| median | 0.54 | 0.56 | 171.5 | 0.7959* |
| PGBD2 | | | | |
| mean ± SD | 1.38 ± 0.71 | 1.04 ± 0.84 | | 0.2366⁺ |
| median | 1.28 | 0.89 | 139.0 | 0.2121* |
| ID3 | | | | |
| mean ± SD | 0.31 ± 0.25 | 0.14 ± 0.08 | | 0.0017⁺ |
| median | 0.23 | 0.13 | 110.5 | 0.0342* |

Furthermore, all the expression values of the nine biomarkers were examined by logistic regression analysis in the SAS software. The analyzed results are shown in table 25.

TABLE 25
The univariate logistic regression results for the nine biomarkers

| | | | 95% confidence | | | | |
|---|---|---|---|---|---|---|---|
| | | | interval | | | | |
| | | Odds | Lower | Upper | | | Cut-off |
| | β-estimate | ratio | bound | bound | P value | AUC | value |
| HIF1A | −0.1377 | 0.871 | 0.455 | 1.668 | 0.6777 | 55.2% | 1.90 |
| FAM84B | −31.2656 | <0.001 | <0.001 | >999.999 | 0.1260 | 68.1% | 0.02 |
| CRIP2 | −0.8584 | 0.424 | 0.152 | 1.183 | 0.1011 | 77.4% | 0.71 |
| GSN | 0.0976 | 1.102 | 0.817 | 1.488 | 0.5239 | 38.9% | 4.95 |
| RPL15 | −0.6216 | 0.537 | 0.163 | 1.765 | 0.3058 | 63.0% | 1.16 |
| DLG1 | −0.4599 | 0.631 | 0.077 | 5.145 | 0.6675 | 52.1% | 0.32 |
| MAT2A | 0.2557 | 1.291 | 0.213 | 7.840 | 0.7812 | 47.0% | 0.61 |
| PGBD2 | −0.7395 | 0.477 | 0.140 | 1.626 | 0.2370 | 64.2% | 1.09 |
| ID3 | −7.5964 | <0.001 | <0.001 | 2.606 | 0.0818 | 74.1% | 0.21 |

The results were further analyzed by Cox proportional hazard model-univariate for survival rate analysis as shown in table 26.

TABLE 26
The results of post-surgery survival rate analyzed by
Cox proportional hazard model-univariate

| | | | 95% confidence | | |
|---|---|---|---|---|---|
| | | | interval | | |
| | Estimate of | | Lower | Upper | |
| | parameter | Hazard ratio | bound | bound | P value |
| HIF1A | −0.12569 | 0.882 | 0.507 | 1.534 | 0.6564 |
| FAM84B | −23.88818 | 0.000 | 0.000 | 8214.765 | 0.1547 |
| CRIP2 | −0.55100 | 0.576 | 0.259 | 1.281 | 0.1763 |
| GSN | 0.16230 | 1.176 | 0.859 | 1.610 | 0.3108 |
| RPL15 | −0.31132 | 0.732 | 0.260 | 2.062 | 0.5554 |
| DLG1 | −0.13355 | 0.875 | 0.126 | 6.083 | 0.8926 |
| MAT2A | 0.60021 | 1.823 | 0.291 | 11.403 | 0.5212 |
| PGBD2 | −0.48326 | 0.617 | 0.198 | 1.918 | 0.4038 |
| ID3 | −5.06602 | 0.006 | 0.000 | 3.310 | 0.1129 |

The results shown in table 26 suggest that the four biomarker comprising FAM84B, GSN, MAT2A and ID3 can provide to predict survival rate of a gastric cancer patient after surgery. Therefore, the expression values of the four biomarkers were further analyzed by Cox proportional hazard model-multivariate model to identify the cut-off values for distinguishing the high risk and low risk as shown in table 27.

TABLE 27
The Cox proportional hazard model-multivariate results
for FAM84B, GSN, MAT2A and ID3

| | | | 95% confidence | | |
|---|---|---|---|---|---|
| | | | Lower | Upper | |
| | Estimate | Hazard rate | bound | bound | P value |
| FAM84B | −98.35996 | 0.000 | 0.000 | 0.051 | 0.0432 |
| GSN | 0.56503 | 1.760 | 1.139 | 2.718 | 0.0109 |
| MAT2A | 4.71969 | 112.133 | 3.181 | 3952.580 | 0.0094 |
| ID3 | −19.52864 | 0.000 | 0.000 | 0.041 | 0.0190 |

The equation is acquired according to the results shown in table 27 as below:

risk score=(0.56503×GSN value)+(4.71969×MAT2A value)−(98.35996×FAM84B value)−(19.52864×ID3 value).

Moreover, the equation was further examined by logistic regression analysis to obtain the statistic results as shown in table 28 and FIG. 12.

TABLE 28
The univariate logistic regression result for risk score equation

| | | | 95% confidence | | | | |
|---|---|---|---|---|---|---|---|
| | | | interval | | | | |
| | | Odds | Lower | Upper | | | Cut-off |
| | Estimate β | ratio | bound | bound | P value | AUC | value |
| Integration | 1.0991 | 3.001 | 1.274 | 7.072 | 0.0120 | 92.4% | −0.04 |
| of risk | | | | | | | |

According to the table 28 and FIG. 12, it suggests that a risk score obtained by introducing the expression values of FAM84B, GSN, MAT2A and ID3 into the equation can be provided to predict the post-surgery survival rate in the gastric cancer patients. While a patient with risk score equal or less than −0.04, the patient is categorized as low-risk population and has better survival rate. Conversely, while a patient with risk score higher than −0.04, the patient is categorized as high-risk population and has worse survival rate. Moreover, the accuracy of predicting post-surgery survival rate by the risk score is 92.4%.

Divided risk score into two groups according to its cut-off value, and the two groups were further examination by logistic regression model and Cox proportional hazard model-multivariate model. The analyzed results are shown in table 29, wherein P value was determined using fisher's exact test.

TABLE 29
The results for the two group of risk score

| | | | | Odds ratio/ | | |
|---|---|---|---|---|---|---|
| | | Survival | Death | hazard ratio | | |
| | Cut-off | n = 36 | n = 8 | (95% confidence | | |
| | value | Cases (ratio, %) | Cases (ratio, %) | interval) | AUC | P value |
| Logistic regression analysis | | | | Odds ratio | | |
| Low risk | <=−0.04 | 33 (91.67) | 2 (25.00) | 1 | | |
| High risk | >−0.04 | 3 (8.33) | 6 (75.00) | 33.0000 (4.5135, | 83.3% | <0.0001 |
| | | | | 241.2772) | | |
| Cox regression model (Cox proportional hazards model) | | | | Hazard ratio | | |
| Low risk | <=−0.04 | 33 (91.67) | 2 (25.00) | 1 | | |
| High risk | >−0.04 | 3 (8.33) | 6 (75.00) | 15.217 (2.996, | | 0.0010 |
| | | | | 77.284) | | |

As shown in table 29, it suggests that the death risk after surgery management in the high-risk patients is 33.0 with the accuracy up to 83.3%. After consideration with the factor of survival time, the death risk after surgery management is 15.2.

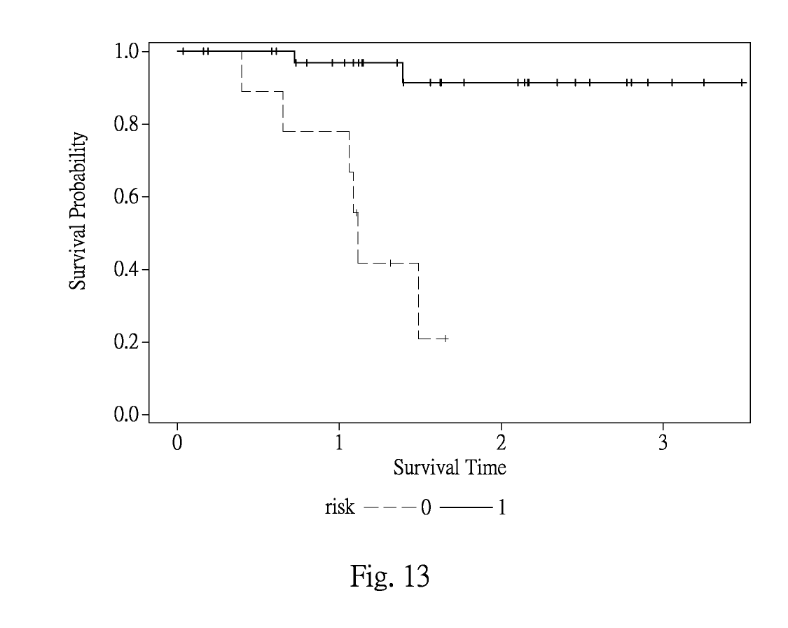

Furthermore, FIG. 13 shows the survival curves of the patients with different risk ranks by Kaplan-Meier, wherein the p value determined using Log-Rank test is less than 0.0001. According to FIG. 13, it suggests the two survival curves with different risk rank have significant difference.

In addition, the nine biomarkers were respectively categorized into two groups according to its cut-off values for the logistic regression analysis in SAS software. The statistic results are shown in table 30, wherein the p value was determined using fisher's exact test.

TABLE 30
The statistic results for the two groups of
each biomarker divided by its cut-off value

| | | Survival | | Death | Odds ratio | | |
|---|---|---|---|---|---|---|---|
| | Cut-off | n = 36 | | n = 8 | (95% confidence | | P |
| | value | Cases (%) | | Cases (%) | interval) | AUC | value |
| HIF1A | ≦1.90 | 21 | (58.33) | 6 (75.00) | 1 | | |
| | >1.90 | 15 | (41.67) | 2 (25.00) | 0.4667 (0.0826, | 58.3% | 0.4546 |
| | | | | | 2.6377) | | |
| FAM84B | ≦0.02 | 9 | (25.00) | 6 (75.00) | 1 | | |
| | >0.02 | 27 | (75.00) | 2 (25.00) | 0.1111 (0.0189, | 75.0% | 0.0126 |
| | | | | | 0.6518) | | |
| CRIP2 | ≦0.71 | 10 | (27.78) | 6 (75.00) | 1 | | |
| | >0.71 | 26 | (72.22) | 2 (25.00) | 0.1282 (0.0221, | 73.6% | 0.0190 |
| | | | | | 0.7442) | | |
| GSN | ≦4.95 | 29 | (80.56) | 6 (75.00) | 1 | | |
| | >4.95 | 7 | (19.44) | 2 (25.00) | 1.3810 (0.2281, | 52.8% | 0.6585 |
| | | | | | 8.3594) | | |
| RPL15 | ≦1.16 | 16 | (44.44) | 6 (75.00) | 1 | | |
| | >1.16 | 20 | (55.56) | 2 (25.00) | 0.2667 (0.0473, | 65.3% | 0.2404 |
| | | | | | 1.5043) | | |
| DLG1 | ≦0.32 | 9 | (25.00) | 3 (37.50) | 1 | | |
| | >0.32 | 27 | (75.00) | 5 (62.50) | 0.5556 (0.1102, | 56.3% | 0.6625 |
| | | | | | 2.8016) | | |
| MAT2A | ≦0.61 | 20 | (55.56) | 4 (50.00) | 1 | | |
| | >0.61 | 16 | (44.44) | 4 (50.00) | 1.2500 (0.2696, | 52.8% | 1.0000 |
| | | | | | 5.7954) | | |
| PGBD2 | ≦1.09 | 16 | (44.44) | 5 (62.50) | 1 | | |
| | >1.09 | 20 | (55.56) | 3 (37.50) | 0.4800 (0.0994, | 59.0% | 0.4485 |
| | | | | | 2.3190) | | |
| ID3 | ≦0.21 | 17 | (47.22) | 7 (87.50) | 1 | | |
| | >0.21 | 19 | (52.78) | 1 (12.50) | 0.1278 (0.0142, | 70.1% | 0.0544 |
| | | | | | 1.1479) | | |

According to the results in table 30, it reveals the expression values of FAM84B and CRIP2 are significantly correlated with the post-surgery survival rate of the patients. Herein, a patient exhibiting FAM84B expression value higher than 0.02 reveals the post-surgery death risk at 0.1111 with the prediction accuracy up to 75% and a patient exhibiting CRIP2 expression value higher than 0.71 reveals the post-surgery death risk at 0.1282 with the prediction accuracy up to 73.6%.

The above results were further analyzed by Cox proportional hazard model-univariate for determining the correlation of survival rate and each biomarker expression value. The statistic results are shown in table 31.

TABLE 31
The results for the two groups of each biomarker divided by
its cut-off value by Cox proportional hazard model-univariate

| | | | 95% confidence interval | | |
|---|---|---|---|---|---|
| | Cut-off | Hazard | Lower | Upper | |
| | value | ratio | bound | bound | P value |
| HIF1A | ≦1.90 | 1 | | | |
| | >1.90 | 0.491 | 0.099 | 2.441 | 0.3845 |
| FAM84B | ≦0.02 | 1 | | | |
| | >0.02 | 0.196 | 0.039 | 0.039 | 0.0461 |
| CRIP2 | ≦0.71 | 1 | | | |
| | >0.71 | 0.246 | 0.050 | 1.226 | 0.0870 |
| GSN | ≦4.95 | 1 | | | |
| | >4.95 | 1.315 | 0.264 | 6.556 | 0.7385 |
| RPL15 | ≦1.16 | 1 | | | |
| | >1.16 | 0.414 | 0.084 | 2.056 | 0.2810 |
| DLG1 | ≦0.32 | 1 | | | |
| | >0.32 | 0.631 | 0.150 | 2.651 | 0.5297 |
| MAT2A | ≦0.61 | 1 | | | |
| | >0.61 | 1.101 | 0.275 | 4.415 | 0.8919 |
| PGBD2 | ≦1.09 | 1 | | | |
| | >1.09 | 0.651 | 0.155 | 2.744 | 0.5590 |
| ID3 | ≦0.21 | 1 | | | |
| | >0.21 | 0.178 | 0.022 | 1.451 | 0.1069 |

The results in table 31 reveal that the cut-off value of FAM84B exhibits the significance correlation with post-surgery survival rate in the patients. Furthermore, the survival curve shown in FIG. 14 is made by Kaplan-Meier according the different rank of FAM84B expression, wherein the p value determined by Log-Rank test is 0.0263 that is less than 0.05. Therefore, the results shown in table 31 and FIG. 14 reveal that the different risk rank calculated by FAM84B expression value exhibits the significant correlation with post-surgery survival rate. According to table 31 and FIG. 14, it is known that a patient with FAM84B expression value higher than 0.02 reveals 0.196 fold of death risk with the comparison of the patients with FAM84B expression value equal or less than 0.02.

According to the embodiments and the examples, any one of the nine biomarkers or combination thereof is capable of diagnosing gastric cancer, such as detection early gastric cancer, staging gastric cancer, diagnosis lymph node metastasis and prediction post-surgery survival rate. Therefore, the method for diagnosis gastric cancer disclosed by the invention is through measuring the expression value of at least one biomarker to determine the occurrence, progression or post-surgery survival rate of the gastric cancer. Based on any one of the nine biomarkers of the invention exhibits the specificity and great sensitivity, so measuring the expression value of at least one biomarker can obtain greater accuracy. Especially, diagnosis gastric cancer by measuring single biomarker can retrench the time and cost.

Moreover, despite detection of mRNA value of the nine biomarkers by RT-PCR, the measurement of protein value of the nine biomarkers is also achieved by the other bioanalyzing methods including ELISA (enzyme-linked immunosorbent assay), EIA (enzyme-linked immunoassay), immunofluorescence staining and western blotting. However, the bioanalyzing methods to detect the biomarker expression is not limiting in scope. In addition, any one of the nine biomarkers or combination thereof can be spotted on the matrix which is microarray platform for determining the biomarker expression in clinical.

Furthermore, comparing the prior art, the present invention using the blood specimen as the sample can be more convenient for the medical staffs to collect from the patient and to improve the volition of patient to detect gastric cancer for preventing in clinical.

The invention claimed is:

1. A method for detecting the risk of early gastric cancer, comprising the following steps:
a. providing at least one biological sample from a subject with gastric cancer and at least one biological sample from a subject without gastric cancer;
b. measuring the expression of at least one biomarker in the biological samples, wherein the biomarker is CRIP2;
c. analyzing the expressions of the biomarker obtained in step b by regression analysis and drawing a receiver operating characteristic (ROC) curve to obtain a cut-off value;
d. measuring the expression of the biomarker in a sample from a test subject; and
e. comparing the expression of the biomarker in the sample from the test subject with the cut-off value of step c to predict the risk of the test subject having early gastric cancer, wherein when the expression value of the biomarker of the test subject below the cut-off value is indicative of a higher risk of the presence of early gastric cancer in the test subject.

2. The method for detecting the risk of early gastric cancer according to claim 1, wherein the sample is selected from the group consisting of a blood specimen, a cell of stomach wall and a tissue of stomach wall.

3. The method for detecting the risk of early gastric cancer according to claim 1, wherein the biomarker of step b further includes a gene selected from the group consisting of FAM84B, RPL15, DLG1, MAT2A, PGBD2 and ID3.

4. The method for detecting the risk of early gastric cancer according to claim 1, wherein the biomarker is further spotted on a matrix.

5. The method for detecting the risk of early gastric cancer according to claim 4, wherein the matrix is a microarray.

6. The method for detecting the risk of early gastric cancer according to claim 1, wherein:
step b: further measuring the expression of another biomarker: HIF1A;
step c: analyzing the expressions of the another biomarker obtained in step b by regression analysis and drawing another ROC curve to obtain another cut-off value;
step d: measuring the expression of the another biomarker in the sample from the test subject; and
step e comparing the expression of the another biomarker in the sample from the test subject with the another cut-off value of step c to predict the risk of the test subject having early gastric cancer, wherein when the expression value of the another biomarker of the test subject above the another cut-off value is indicative of a higher risk of the presence of early gastric cancer in the subject.


SEQUENCE LISTING (18 sequences)

SEQ ID NO: 1 - DNA, 21 bp, Artificial Sequence
  Left primer of gene CRIP2
ccctgctatggaatcctcttc

SEQ ID NO: 2 - DNA, 18 bp, Artificial Sequence
  Right primer of gene CRIP2
ggggtcccggtcatagat

SEQ ID NO: 3 - DNA, 20 bp, Artificial Sequence
  Left primer of gene DLG1
gaactagccagaagcgatcc

SEQ ID NO: 4 - DNA, 24 bp, Artificial Sequence
  Right primer of gene DLG1
cagaagcattaataacatggagga

SEQ ID NO: 5 - DNA, 20 bp, Artificial Sequence
  Left primer of gene FAM84B
ccttcaaactcgccctagaa

SEQ ID NO: 6 - DNA, 19 bp, Artificial Sequence
  Right primer of gene FAM84B
cagggtcatttgcacaggt

SEQ ID NO: 7 - DNA, 18 bp, Artificial Sequence
  Left primer of gene GSN
catctcgcgcatgcagta

SEQ ID NO: 8 - DNA, 21 bp, Artificial Sequence
  Right primer of gene GSN
cttgaagatgggactctcacg

SEQ ID NO: 9 - DNA, 20 bp, Artificial Sequence
  Left primer of gene HIF1A
tggaatggagcaaaagacaa

SEQ ID NO: 10 - DNA, 20 bp, Artificial Sequence
  Right primer of gene HIF1A
tggtcagctgtggtaatcca

SEQ ID NO: 11 - DNA, 19 bp, Artificial Sequence
  Left primer of gene ID3
cgtgtcctgacacctccag

SEQ ID NO: 12 - DNA, 21 bp, Artificial Sequence
  Right primer of gene ID3
ggatttggtgaagtcaagtgg

SEQ ID NO: 13 - DNA, 24 bp, Artificial Sequence
  Left primer of gene MAT2A
acagaaatgaagaagacattggtg

SEQ ID NO: 14 - DNA, 20 bp, Artificial Sequence
  Right primer of gene MAT2A
tcatcagtggcatagccaaa

SEQ ID NO: 15 - DNA, 20 bp, Artificial Sequence
  Left primer of gene PGBD2
ggggtagggcaggttcttag

SEQ ID NO: 16 - DNA, 20 bp, Artificial Sequence
  Right primer of gene PGBD2
ggatacctctcccagcaatg

SEQ ID NO: 17 - DNA, 21 bp, Artificial Sequence
  Left primer of gene RPL15
ccagctaaagtttgctcgaag

SEQ ID NO: 18 - DNA, 20 bp, Artificial Sequence
  Right primer of gene RPL15
gactctcagagccccacagt